(19) United States
(12) Patent Application Publication
(10) Pub. No.: US 2024/0318114 A1
(43) Pub. Date: Sep. 26, 2024

(54) BIDIRECTIONAL TANGENTIAL FLOW FILTRATION (TFF) PERFUSION SYSTEM

(71) Applicant: Just-Evotec Biologics, Inc., Seattle, WA (US)

(72) Inventors: Anastasia Nicolov, Shoreline, WA (US); Myles Daniel Hollenbach, Everett, WA (US); Christopher J. Ford, Redmond, WA (US)

(73) Assignee: Just-Evotec Biologics, Inc., Seattle, WA (US)

(21) Appl. No.: 18/579,358

(22) PCT Filed: Jul. 11, 2022
(86) PCT No.: PCT/US2022/036690

§ 371 Date: Jan. 14, 2024

Related U.S. Application Data
(60) Provisional application No. 63/222,210, filed on Jul. 15, 2021.

(51) Int. Cl. C12M 1/00; B01D 61/22; C12M 1/36

(52) U.S. Cl. CPC C12M 29/10; B01D 61/22; C12M 29/16; C12M 41/48; B01D 2311/2523; B01D 2311/2688; B01D 2313/243; B01D 2315/10; B01D 2321/205

(57) ABSTRACT

Disclosed is an automated tangential flow filtration (TFF) perfusion bioreactor for culturing eukaryotic cells, useful for manufacturing a purified protein of interest, such as, but not limited to, a recombinant or naturally occurring protein and/or a therapeutic or other medically useful protein. The disclosed TFF perfusion bioreactor system can operate at high cell densities for long production cultivation periods (13-90 days) that provide the greatest efficiencies, whether in batch mode, continuous, or semi-continuous biologics manufacturing platforms.

### BACKGROUND OF THE INVENTION

### 1. Field of the Invention

This invention relates to the field of automated manufacturing facilities and processes for the production of therapeutic proteins.

### 2. Discussion of the Related Art

The biopharmaceutical industry is undergoing major changes, prompted in part by the surge in approvals of new biotherapeutics, higher protein expression rates and increased pressure from the biosimilars market. (Levine et al., Efficient, flexible facilities for the 21st century, BioProcess International 10(11):20-30 (2012)).

An expected surge in the pharmaceutical market share of biologics (from 11% in 2002 to around 20% in 2017), coupled with the need for affordable medicine access in developing regions of the world requires the development of fast, sustainable and cost-effective manufacturing methods. (Walsh, Biopharmaceutical benchmarks 2014, Nature biotechnology 32(10):992-1002 (2014)).

Consequently, the field of biologics manufacturing technology has advanced in alternative directions to capitalize on key advantages such as higher throughput, operational flexibility and cost savings, as well as footprint reduction and reduced environmental impact. For example, modern cell lines and media have been engineered to target higher cell densities, especially when contrasted with fed-batch processing, with some cultures achieving viable cell densities greater than 100 million cells/mL. (Clincke et al., Very high density of chinese hamster ovary cells in perfusion by alternating tangential flow or tangential flow filtration in wave Bioreactor™—part ii: Applications for antibody production and cryopreservation, Biotechnology Progress 29(3):768-777 (2013)).

In the culture of eukaryotic cells, e.g., mammalian, insect, algal, or yeast cell lines, for production of protein-based pharmaceutical products, bioreactors are often used as culture vessels in order to control process variables within a desired range and easily scale up from small volumes to manufacturing scale. Perfusion systems are some of the most efficient bioreactor culturing methods. In such perfusion bioreactor systems, cells are fed fresh, nutrient-rich culture medium on a continuous or semi-continuous basis, while spent medium containing the harvested protein products (known as the “harvest cell culture fluid”) is continually pulled out. (See, e.g., Warikoo et al., Integrated continuous production of recombinant therapeutic proteins, Biotechnology and Bioengineering 109(12):3018-3029 (2012); Godawat et al., End-to-end integrated fully continuous production of recombinant monoclonal antibodies, Journal of Biotechnology 213:13-19 (2015); Vandiver et al., Automated Biomanufacturing Systems, Facilities, and Processes, WO2020/168315A1; Farid et al., Evaluating the economic and operational feasibility of continuous processes for monoclonal antibodies, Continuous Processing in Pharmaceutical Manufacturing pp. 433-456 (2015); Kelley, Industrialization of mAb production technology: the bioprocessing industry at a crossroads, mAbs 1(5):443-452 (2009); Croughan et al., The future of industrial bioprocessing: Batch or continuous?, Biotechnology and Bioengineering 112:648-651 (2015); Pollock et al., Fed-batch and perfusion culture processes: Economic, environmental, and operational feasibility under uncertainty, Biotechnology and Bioengineering 110(1):206-219 (2013); Konstantinov et al., White paper on continuous bioprocessing, Journal of Pharmaceutical Sciences 104(3):813-820 (2015)).

For the cells to be retained in the bioreactor, the culture is filtered using a perfusion filter. Commonly, hollow fiber filters are used; culture containing cells is continually run through the fiber lumens, while filtrate (also known as “permeate”) is pulled out “perpendicular” to the culture flow, through pores too small to pass cells.

Two common systems used for perfusion are Alternating Tangential Flow (ATF) filtration and Tangential Flow Filtration (TFF). Both are typically used in the biologics industry and have unique advantages and disadvantages. For example, ATF is claimed to be gentler on cells, and its alternating flow direction through the perfusion filter helps prevent debris build-up in the fibers. However, ATF flow rates are relatively slow and result in high residence times of cells in the filter and outside of the controlled environment of the bioreactor. This may result in viability and productivity declines. Furthermore, the diaphragm pumps of ATF tend not to maintain flow well in high-density (e.g., >80×10⁶ cells/mL), viscous cultures.

Industry-standard TFF systems flow culture in a single direction, using a pump such as (but not limited to) a peristaltic pump or a centrifugal pump. The TFF systems generally have higher shear than ATF due to the pump types used and higher crossflow rates, though shear is dependent on pump type and can vary greatly. While low-shear TFF systems are suitable for cultures at high densities (because of their low residence time and ability to maintain flow at higher viscosities), filter fouling can be a significant issue, with buildup of cells and debris on one side of the filter, plugging fibers and reducing filter lifetime. Higher-shear TFF systems can exacerbate these issues by causing declines in viability and increases in cell debris in the culture.

Some workers have attempted various solutions to the problem of filter fouling in TFF perfusion bioreactor systems in various ways. (See, e.g., Zhou et al., Methods and Systems for Processing a Cell Culture, U.S. patent application 2015158907A1 and WO2015/039115A1; Wales et al., Reversible Liquid Filtration System, U.S. patent application 20190241856A1 and WO2018/015386A1; Lu et al., Perfusion Bioreactor with Filtration Systems, U.S. patent application 2019338238 A1 and WO2019/213567A1).

However, none of these solutions has been satisfactory for TFF perfusion bioreactor systems operating at high cell densities for the long production cultivation periods that provide the greatest efficiencies, whether employed in batch mode, continuous, or semi-continuous biologics manufacturing platforms.

The present invention provides solutions to these challenges, meets this need and is fully compatible with an integrated biologics manufacturing facility.

### SUMMARY OF THE INVENTION

The present invention relates to an automated tangential flow filtration (TFF) perfusion bioreactor for culturing eukaryotic cells, useful in facilities for manufacturing purified proteins of interest, e.g., therapeutic, prophylactic, diagnostic, or other medically valuable proteins, and drug substances.

The inventive automated TFF perfusion bioreactor has an inlet for periodically receiving volumes of fresh liquid cell culture medium and is configured with a chamber for culturing eukaryotic cells (e.g., mammalian cells, insect cells, algal cells, plant cells, or yeast cells). The inventive automated TFF bioreactor comprises:
(i) a perfusion system fluidly linked in a recirculation loop with the bioreactor through a first port of the recirculation loop and a second port of the recirculation loop; the recirculation loop includes, between the first port and the second port, a hollow fiber filter comprising a lumen having a porous surface capable of preventing the passage of whole cells but allowing volumes of conditioned cell culture medium to pass and to exit the perfusion system into a permeate receptacle, wherein volumes of conditioned medium containing cells from the bioreactor can enter through the first port into the recirculation loop, and flow tangentially in relation to the porous surface, and a retentate can flow out from the recirculation loop through the second port back into the bioreactor; and
(ii) a low-shear pump system comprising at least one pump head that directs the flow of the conditioned medium containing cells through the recirculation loop in a first direction through the lumen(s) of the hollow fiber filter and, interchangeably, in a second direction, opposite the first direction. In one preferred embodiment, the low shear pump system comprises one or more centrifugal pumps.

The inventive automated TFF bioreactor also comprises a controller capable of automatically switching the low-shear pump system to direct the flow of the conditioned medium containing cells through the recirculation loop in the first direction, and after a predetermined period (e.g., about 20 minutes, about 1 hour, about 6 hours, about 12 hours, up to about 24 hours), automatically switching the low-shear pump system to direct the flow of the conditioned medium containing cells to the second direction, and vice versa, wherein the first and the second directions are periodically exchanged and the conditioned medium containing cells synchronously enters the recirculation loop from the bioreactor through the second port instead of the first port and the retentate returns to the bioreactor through the first port instead of the second port, and vice versa. The inventive automated TFF perfusion bioreactor is configured to operate over a production cultivation period of 13-90 days at a viable cell density of about 60-300×10⁶/mL, and compared to an equivalently-scaled non-switching system, sieving by the hollow fiber filter is significantly improved.

The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features is not found together in the same sentence, or paragraph, or section of this document. In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1A shows a schematic representation of a prior art TFF perfusion bioreactor having a recirculation loop with a unidirectional flow of conditioned culture medium directed by a pump.

FIG. 1B shows a schematic representation of an embodiment of the automated tangential flow filtration (TFF) perfusion bioreactor of the present invention. In the embodiment illustrated in FIG. 1B, two low-shear (e.g., centrifugal) pump systems (designated “pump”) are shown, each pump system comprising a pump head dedicated to directing flow through the recirculation loop in the first direction or in the second direction, respectively, after a predetermined period. In other embodiments, a single low-shear (e.g., centrifugal) pump system can be used that has two pump heads (one pump head is dedicated to directing flow in the first direction and the other pump head is dedicated to directing flow in the second direction).

FIG. 2 shows viable cell densities (×10⁶ cells/mL) measured in the experiment described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 3 shows packed cell volume (PCV; %) measured in the experiment described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 4 shows cell viability (%) measured in the experiment described in Example 1. The data points for the bidirectional condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 5 shows viable cell diameters (μm) measured in the experiment described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 6 shows sieving (%) measured under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 7 shows transmembrane pressure (TMP; bars) under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 8 shows productivity (g/L/d) for an antibody protein of interest, measured in the experiment described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 9 shows specific productivity (pg/cell/d) for an antibody protein of interest, measured in the experiment described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 10 shows lactate concentration (g/L) measured in the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 11 shows ammonium concentration (g/L) measured in the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 12 shows totalized lactate dehydrogenase (LDH) enzyme measured (U/L) in the conditioned culture medium as an indicator of cell death under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 13 shows glucose concentration (g/L) measured in the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 14 shows pH measured in the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 15 shows cumulative base addition (mL of 1 M sodium carbonate) to the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 16 shows dissolved carbon dioxide (mm Hg) measured in the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 17 shows osmolality (mOsm/kg) measured in the conditioned culture medium under the experimental conditions described in Example 1. The data points for the bidirectional flow condition with a 10-minute cycle time are represented with circles; data points for the bidirectional flow condition with a 20-minute cycle time are represented with squares, and the data points for the bidirectional flow condition with a 60-minute cycle time are represented with triangles. The data points for the unidirectional flow condition are represented with asterisks, and data points for the peristaltic pump-driven condition are represented by “X”s.

FIG. 18 shows viable cell densities (×10⁶ cells/mL) measured in the experiment described in Example 2. Data points for the bidirectional flow condition are represented with asterisks, and the data points for the unidirectional flow condition are represented with squares.

FIG. 19 shows packed cell volume (PCV; %) measured in the experiment described in Example 2. Data points for the bidirectional flow condition are represented with asterisks, and the data points for the unidirectional flow condition are represented with squares.

FIG. 20 shows cell viability (%) measured in the experiment described in Example 2. Data points for the bidirectional flow condition are represented with asterisks, and the data points for the unidirectional flow condition are represented with squares.

FIG. 21 shows viable cell diameters (μm) measured in the experiment described in Example 2. Data points for the bidirectional flow condition are represented with asterisks, and the data points for the unidirectional flow condition are represented with squares.

FIG. 22 shows sieving (%) measured under the experimental conditions described in Example 2. Data points for the bidirectional flow condition are represented with asterisks, and the data points for the unidirectional flow condition are represented with squares.

FIG. 23 shows transmembrane pressure (TMP; bars) under the experimental conditions described in Example 2. Data points for the bidirectional flow condition are represented with asterisks, and the data points for the unidirectional flow condition are represented with squares.

FIG. 24 shows productivity (g/L/d) for an antibody protein of interest, measured in the experiment described in Example 2. Data points for the bidirectional flow condition are represented with asterisks, and the data points for the unidirectional flow condition are represented with squares.

FIG. 25 shows cell-specific productivity (pg/cell/d) for an antibody protein of interest, measured in the experiment described in Example 2. Data points for the bidirectional flow condition are represented with asterisks, and the data points for the unidirectional flow condition are represented with squares.

FIG. 26 shows lactate concentration (g/L) measured in the conditioned culture medium under the experimental conditions described in Example 2. Data points for the bidirectional flow condition are represented with asterisks, and the data points for the unidirectional flow condition are represented with squares.

FIG. 27 shows ammonium concentration (g/L) measured in the conditioned culture medium under the experimental conditions described in Example 2. Data points for the bidirectional flow condition are represented with asterisks, and the data points for the unidirectional flow condition are represented with squares.

FIG. 28 shows totalized lactate dehydrogenase (LDH) enzyme measured (U/L) in the conditioned culture medium as an indicator of cell death under the experimental conditions described in Example 2. Data points for the bidirectional flow condition are represented with asterisks, and the data points for the unidirectional flow condition are represented with squares.

FIG. 29 shows glucose concentration (g/L) measured in the conditioned culture medium under the experimental conditions described in Example 2. Data points for the bidirectional flow condition are represented with asterisks, and the data points for the unidirectional flow condition are represented with squares.

FIG. 30 shows pH measured in the conditioned culture medium under the experimental conditions described in Example 2. Data points for the bidirectional flow condition are represented with asterisks, and the data points for the unidirectional flow condition are represented with squares.

FIG. 31 shows cumulative base addition (mL of 1 M sodium carbonate) to the conditioned culture medium under the experimental conditions described in Example 2. Data points for the bidirectional flow condition are represented with asterisks, and the data points for the unidirectional flow condition are represented with squares.

FIG. 32 shows dissolved carbon dioxide (mm Hg) measured in the conditioned culture medium under the experimental conditions described in Example 2. Data points for the bidirectional flow condition are represented with asterisks, and the data points for the unidirectional flow condition are represented with squares.

FIG. 33 shows osmolality (mOsm/kg) measured in the conditioned culture medium under the experimental conditions described in Example 2. Data points for the bidirectional flow condition are represented with asterisks, and the data points for the unidirectional flow condition are represented with squares.

FIG. 34 shows viable cell densities (×10⁶ cells/mL) measured in the experiment described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 35 shows packed cell volume (PCV; %) measured in the experiment described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 36 shows cell viability (%) measured in the experiment described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 37 shows viable cell diameters (μm) measured in the experiment described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 38 shows sieving (%) measured under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 39 shows transmembrane pressure (TMP; bars) under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 40 shows productivity (g/L/d) for an antibody protein of interest, measured in the experiment described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 41 shows cell-specific productivity (pg/cell/d) for an antibody protein of interest, measured in the experiment described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 42 shows lactate concentration (g/L) measured in the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 43 shows ammonium concentration (g/L) measured in the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 44 shows totalized lactate dehydrogenase (LDH) enzyme measured (U/L) in the conditioned culture medium as an indicator of cell death under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 45 shows glucose concentration (g/L) measured in the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 46 shows pH measured in the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 47 shows cumulative base addition (mL of 1 M sodium carbonate) to the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 48 shows dissolved carbon dioxide (mm Hg) measured in the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

FIG. 49 shows osmolality (mOsm/kg) measured in the conditioned culture medium under the experimental conditions described in Example 3. Data points are represented as follows: 1-hour bidirectional condition is represented with triangles; 6-hour bidirectional condition is represented with circles; 12-hour bidirectional condition is represented with squares; and the unidirectional condition is represented with “X”s.

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes one protein or a plurality of proteins; reference to “a bioreactor” includes one bioreactor or a plurality of bioreactors.

The present invention is directed to an automated tangential flow filtration (TFF) perfusion bioreactor, which is useful as a part of, and compatible with, an integrated, continuous or semi-continuous, and automated process for manufacturing a purified protein of interest (for example, but not limited to, a purified protein drug substance). If desired, one or more of the inventive automated TFF perfusion bioreactors can be used in any particular process for manufacturing a purified protein of interest. The bioreactor of the inventive automated TFF perfusion bioreactor can be configured for single-use, if desired, and such a single-use bioreactor can be disposable after a single production cultivation period, or alternatively, the bioreactor can be re-sterilizable and reusable. Similarly, the recirculation loop containing the filter and elements of the perfusion system can be reusable or configured for single use. Typically, such biologics manufacturing processes are performed under aseptic operational conditions and involve automation-controlled regulation of flow rates in both upstream and downstream unit operations. The various steps of the manufacturing process can be performed within an automated facility, either in a single cleanroom or a plurality of separate modular cleanrooms, which can, optionally, be automation-controlled, and, if multiple automated TFF perfusion bioreactors of the invention are employed, they can be placed within a single cleanroom or in a plurality of separate cleanrooms, as desired.

In perfusion bioreactor operation, there are single-use and reusable options at all industrially applicable bioreactor scales, from bench-top to large-scale manufacturing. Commonly, benchtop bioreactors are made of glass and stainless steel (e.g., commercially available bioreactors by Applikon Biotechnology B.V. and other manufacturers), which can be sterilized by autoclave and reused hundreds, if not thousands of times. To such reusable bioreactor systems, recirculation loops are attached in an aseptic manner using parts that are specifically designed to attach two sterile units without breaking the sterile envelope of either unit. The recirculation loop, containing the centrifugal pump head(s), the hollow fiber filter of the TFF perfusion system, and various connecting parts and tubing, can contain reusable metal or plastic parts, or it can be entirely single-use and disposable, if desired.

At large scale, various sizes of Xcellerex® brand (Cytiva) single-use bioreactors (SUBs) are commonly used, but other commercially available varieties of SUBs are also useful. In some embodiments the bioreactor (aside from certain probes) is entirely single-use. As with small-scale bioreactors, the recirculation loop can be attached to the bioreactor aseptically, and can, optionally, contain reusable metal or plastic parts (e.g., some hollow fiber filter arrangements commercially available from Asahi-Kasei or other manufacturers), in which case the assembly is autoclaved before attaching to the bioreactor. Alternatively, the recirculation loop containing the hollow fiber filters can be configured entirely for single-use and can usefully arrive gamma-irradiated from the supplier (e.g., from Spectrum®; Repligen, or other manufacturers), ready to attach to the bioreactor. In some useful embodiments, commercially available single-use centrifugal pumps and pump heads are employed (e.g., PuraLev® single-use magnetic drive centrifugal pump kits; Levitronix®).

Across all industrially applicable bioreactor scales, the bioreactor and the recirculation loops of the TFF perfusion system are physically separate from their respective control systems. For the bioreactor, the digital control unit (DCU) regulates pH, dissolved oxygen (DO), temperature, stir speed, and other operating parameters of the culture. For the TFF perfusion system (such as, but not limited to, commercially available Levitronix® or Spectrum® Krosflo® controllers), the flow through the recirculation loop and to the permeate receptacle can be monitored and controlled through flow meters, pressure sensors, and pumps. For both the bioreactor and the perfusion system, the control systems typically remain in place in the lab, or in the manufacturing space, or, if desired, in another electronically accessible remote location, and are typically reused for every experiment or supply run.

Typically, biologics manufacturing processes are performed under aseptic operational conditions and involve automation-controlled regulation of chromatography system flow rates. The various steps of the manufacturing process can be performed within an automated facility, either in a single cleanroom or a plurality of separate modular cleanrooms, which can, optionally, be automation-controlled, and, if multiple automated TFF perfusion bioreactors of the invention are employed, they can be placed within a single cleanroom or in a plurality of separate cleanrooms, as desired.

The term “integrated,” in connection with a process for manufacturing a purified protein of interest (e.g., but not limited to, a protein drug substance), means that one or more upstream steps and/or downstream steps in the manufacturing process are performed under common or coordinated control, based on programmed commands as modified by current sensory feedback of defined parameters in relation to setpoints or flow rates. The term “coordinated” means that two or more operations, steps, processes, components, or systems, are controlled, regulated, or scheduled in a relationship that will ensure efficiency or harmony of their functioning toward a single purpose.

“Upstream” processes include, but are not limited to, e.g., culturing the recombinant host cells; removing cells from the permeate; and fluidly feeding volumes of cell-free permeate from one or more automated TFF perfusion bioreactor(s) of the invention into a permeate receptacle, such as, but not limited to, a single-use surge vessel. “Downstream” process steps can include, but are not limited to, e.g., product capture and purification in a first chromatography system; switching the protein isolate fraction into a viral inactivation system, wherein the viral inactivation system is a low pH or detergent viral inactivation system and, if needed (e.g., in low pH viral inactivation system embodiments), a neutralization system; introducing the virally inactivated product pool into a second chromatography system; switching the purified product pool into a third chromatography system and/or a viral filtration system; and/or switching virus-free filtrate into an ultrafiltration/diafiltration system.

A “continuous” format of a manufacturing process or system means a processing modality wherein a perfusion bioreactor is fluidly connected to a continuous capture chromatography step (e.g., processing by a first chromatography system) in an uninterrupted flow coming from the bioreactor (directly or indirectly via intervening unit operations) to the first chromatography system, which is followed by, and fluidly connected in an uninterrupted flow to, a downstream viral inactivation step, and optionally, in an uninterrupted flow to depth filtration. Further downstream product purification steps (e.g., a second chromatography system, an optional third chromatography system, viral filtration, and processing by ultrafiltration/diafiltration) are fluidly connected, all in an uninterrupted flow to the afore-mentioned upstream processing steps and successively to each other, with optional intervening surge vessels.

A “semi-continuous” format of a manufacturing process means a processing modality wherein a perfusion bioreactor is fluidly connected to a continuous capture chromatography step (e.g., processing by a first chromatography system) in an uninterrupted flow, and to further downstream processing, e.g., by an optional viral inactivation system, and/or, optionally, in an uninterrupted flow downstream to depth filtration, and then storage of product pool in a holding vessel (HV1). Temporary storage of the product pool in the holding vessel is subsequently followed by one or more batch downstream processing step(s), which step(s) can be successively fluidly connected to each other in an uninterrupted flow, e.g., a second chromatography system, an optional third chromatography system, and processing by ultrafiltration/diafiltration, with optional intervening surge vessels or holding vessels (i.e., holding vessels if there are two or more batch steps or operations), as the case may be.

A “perfusion bioreactor” is a bioreactor for culturing cells in which equivalent volumes of culture medium can be added and removed from the reactor while the cells are retained in the bioreactor. A perfusion bioreactor includes a bioreactor and an operably attached perfusion system, which provides a steady source of fresh nutrient medium and removal of cell waste products. The bioreactor and the perfusion system of the perfusion bioreactor can be separate mechanical units that operate in coordination. Numerous commercially available examples include, but are not limited to, a variety of single-use bioreactors (SUBs), commercially available under Xcellerex® brand (Cytiva), Applikon, Broadley-James, or Sartorius brands. In other embodiments, glass and stainless steel autoclavable bioreactors with corresponding control units can be suitably combined by the skilled practitioner with Levitronix® brand or KrosFlo® brand (Spectrum®; Repligen) perfusion control systems and compatible recirculation loop assemblies from Spectrum®, Asahi-Kasei, or other manufacturers. Alternatively, the bioreactor and the recirculation loop can be assembled into a single closed system regulated by a single control unit, for example, but not limited to, perfusion bioreactors that are commercially available under the 3D Biotek brand (Sigma-Aldrich) or KrosFlo® brand perfusion flow-path assemblies and systems (Spectrum®; Repligen), which bioreactors and perfusion systems can be suitably combined into a perfusion bioreactor by the skilled practitioner. Secreted protein products in the bioreactor can be continuously harvested by microfiltration during the process of removing medium via the perfusion system, the protein of interest thus being isolated in a microfilter permeate exiting the perfusion system.

Liquids, such as a culture medium fluid, are subjected to shear forces passing through flow circuit elements such as tubing, pipes, filters, pumps, and connectors. Some liquids (such as emulsions, non-Newtonian liquids, blood and other biological fluids, and fluids containing living cells) include elements that are sensitive to shear stress. A system or environment can be termed “low-shear” if it minimizes the shear stress experienced by these elements, including cellular elements; this is sometimes desired, for example, in the manufacture of biologics.

A “low-shear pump” is a device that can move a shear-sensitive fluid (such as a fluid containing living cells) in a way that minimizes the shear stress on the cells such that they experience little or no damage in transit through the pump. A useful example of a “low-shear” pump is a centrifugal pump. “Centrifugal pumps” are used to transport fluids by the conversion of rotational kinetic energy to the hydrodynamic energy of fluid flow. The rotational energy typically comes from an engine, electric motor, or magnetic drive. The fluid enters the pump impeller along or near to the rotating impeller axis and is accelerated by the impeller, flowing radially outward into a diffuser or volute chamber (casing), from which it exits. For purposes of the present invention, examples of useful centrifugal pumps include, but are not limited to the PuraLev® magnetically-levitated centrifugal pump series (Levitronix®).

A “pump head” is the part of the pump device which contains the mechanism that propels the liquid from the inlet of the pump to the outlet of the pump.

A step of a biologics manufacturing process or a system within an automated manufacturing facility is performed “fluidly,” or is “fluidly connected” or “fluidly linked” (used interchangeably herein) to, or “fluidly receives” material from, another step of the manufacturing process or from another system, when material containing the protein of interest flows by pipe, tubing, or other closed conduit between steps or systems without manual loading or unloading. For example, the perfusion system is fluidly linked in a recirculation loop with the bioreactor in the inventive automated TFF perfusion bioreactor. A step of a manufacturing process or a system within an automated manufacturing facility is commonly called a “unit operation.” A unit operation configured to communicate (e.g., by hard-wiring or wireless connection) with an OPC server is called a “batch unit” or “skid.” Typically, but not necessarily, single-use or reusable bioreactor(s), perfusion system(s), first chromatography system(s), second chromatography system(s), optional third chromatography system(s), and ultrafiltration/diafiltration system(s) are configured as “skids.” Viral inactivation system(s) can also be included in a skid, if desired. (See, e.g., Vandiver et al., Automated Biomanufacturing Systems, Facilities, and Processes, WO2020/168315A1). A unit operation that is controlled not via hard-wiring, but rather via a dongle and/or a Profibus device, or similar digital information storage device and electronic hardware connector(s), is called a “non-batch unit.” For convenience and flexibility, filter banks, heat exchangers, surge vessels, feed tanks, reservoirs, holding vessels, collection vessels or collection tanks (e.g., an elution collection vessel), and portables mixers and other mixing vessels, when optionally present, are typically configured as non-batch units, although unit operations such as these may also be included in a “skid,” involving control via hard-wiring or wireless connection.

The terms “automated,” “automation-controlled,” or “automatically,” are used interchangeably, in connection with a process, facility, or apparatus, such as the inventive automated tangential flow filtration perfusion bioreactor, and refer to computer-control of the implementation or performance of one or more process steps or the operation of a component or a system (e.g., a low-shear or centrifugal pump system), optionally, with attendant feedback regulation of the process step or operation. Typically, an electronic computerized “controller” or “digital control unit,” terms used interchangeably herein, receives input digital signals from one or more sensors or detectors of the physical or chemical parameter to be controlled, and/or from a chronometer or clock, and, in comparison to a predetermined control setpoint (often assigned by the operator using a user interface), the controller determines the necessary output signal required to correct the input value in the direction of the setpoint and issues responsive digital instructions to a system or subsystem.

The term “switching,” or “switch,” used herein interchangeably), with respect to the low-shear or centrifugal pump and the recirculation loop that fluidly links the perfusion system with the bioreactor of the inventive automated TFF perfusion bioreactor, means to change the direction of flow of the cell-containing medium through the recirculation loop from the first direction to the second direction, or vice versa. Such switching can be under the automatic control and regulation of a computer and/or mechanism(s) (e.g., valves or pumps) governed by an external control system, or can be manually controlled and regulated. “Automatically switching” means that the switch in direction does not require manual input on the operator's part, but is controlled instead by the controller, which performs the switch in flow direction on a predetermined cycle time. A “non-switching system” is a TFF perfusion bioreactor system in which the direction of flow through the recirculation loop is not periodically switched.

The term “switching,” or “switch,” used herein interchangeably), with respect to a protein isolate fraction, a purified product pool, a virus-free filtrate, or another pool, fraction, eluate, or resultant liquid outflow from a downstream process step or facility component, means to direct, shunt, steer, stream, or convey that outflow fluidly into a subsequent process step or facility component. Such switching can be under the automatic control and regulation of a computer and/or robotic mechanism(s) (e.g., valves or pumps), or can be manually controlled and regulated.

The term “switchable” in connection with collection vessels, surge vessels, holding vessels, mixing vessels, tanks, bags, conduits, pipe, tubing, or other conveyance, into or out of, or by which liquids can flow, means that such flow can be switched, directed, shunted, steered, streamed, or conveyed fluidly to a different vessel, tank, conduit, pipe, tubing, or conveyance. Such switching can be under the automatic control and regulation of a computer and/or robotic mechanism(s) (e.g., valves or pumps), or can be manually controlled and regulated.

The term “therapeutic protein” means a pharmacologically active protein applicable to the prevention, treatment, or cure of a disease or condition of human beings. Examples of therapeutic proteins include, but are not limited to, monoclonal antibodies, recombinant forms of a native protein (e.g., a receptor, ligand, hormone, enzyme or cytokine), fusion proteins, peptibodies, and/or a monomer domain binding proteins, e.g., based on a domain selected from LDL receptor A-domain, thrombospondin domain, thyroglobulin domain, trefoil/PD domain, VEGF binding domain, EGF domain, Anato domain, Notch/LNR domain, DSL domain, integrin beta domain, and Ca-EGF domain. The preceding are merely exemplary, and a therapeutic protein can comprise any clinically relevant polypeptide target moiety or polypeptide ligand. The term “derivative,” when used in connection with therapeutic proteins of interest, refers to proteins that are covalently modified by conjugation to therapeutic or diagnostic agents, labeling (e.g., with radionuclides or various enzymes), covalent polymer attachment such as PEGylation (derivatization with polyethylene glycol) and insertion or substitution of natural or non-natural amino acids.

A “drug substance” is an active pharmaceutical ingredient (API) intended to furnish pharmacologic activity or other direct effect in the diagnosis, cure, mitigation, treatment, or prevention of disease or to affect the structure or any function of the body. A drug substance can be further formulated, or re-formulated, with buffers, carriers, and/or excipients, and the drug substance can be further dosed in a drug product configuration suitable and/or approved for clinical use.

The term “purify” or “purifying” a desired protein means increasing the degree of purity of the desired protein from a composition or solution comprising the protein of interest (i.e., the “POI,” e.g., a therapeutic or other medically useful protein) and one or more contaminants by removing (completely or partially) at least one contaminant from the composition or solution. An “isolated” protein is one that has been identified and separated from one or more components of its natural environment or of a culture medium in which it has been secreted by a producing cell. In some embodiments, the isolated protein is substantially free from proteins or polypeptides or other contaminants that are found in its natural or culture medium environment that would interfere with its therapeutic, diagnostic, prophylactic, research or other use. “Contaminant” components of its natural environment or medium are materials that would interfere with industrial, research, therapeutic, prophylactic, or diagnostic or uses for the protein of interest, and may include enzymes, hormones, and other proteinaceous or nonproteinaceous (e.g., polynucleotides, lipids, carbohydrates) solutes. Typically, an “isolated protein” or, interchangeably, “protein isolate,” constitutes at least about 5%, at least about 10%, at least about 25%, or at least about 50% of a given sample. In some embodiments, the isolated protein of interest will be “purified”: (1) to greater than 95% by weight of protein, and most preferably, more than 99% by weight, or (2) to homogeneity by SDS-PAGE, or other suitable technique, under reducing or nonreducing conditions, optionally using a stain, e.g., Coomassie blue or silver stain. An isolated naturally occurring antibody includes the antibody in situ within recombinant cells since at least one component of the protein's natural environment will not be present. Typically, however, the isolated or purified protein of interest (e.g., a purified protein drug substance) will be prepared by at least one purification step.

A protein of interest, such as a therapeutic or other medically useful protein, for purposes of the present invention, whether it includes a variant or parental antibody amino acid sequence, is typically produced by recombinant expression technology, although it can also be a naturally occurring protein.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, proteins can be derivatized as described herein and by other well-known organic chemistry techniques.

The term peptide or protein “analog” refers to a polypeptide having a sequence that differs from a peptide sequence existing in nature by at least one amino acid residue substitution, internal addition, or internal deletion of at least one amino acid, and/or amino- or carboxy-terminal end truncations, or additions). An “internal deletion” refers to absence of an amino acid from a sequence existing in nature at a position other than the N- or C-terminus. Likewise, an “internal addition” refers to presence of an amino acid in a sequence existing in nature at a position other than the N- or C-terminus.

A “variant” of a polypeptide (e.g., of an immunoglobulin, or an antibody, or a fusion protein) comprises an amino acid sequence wherein one or more amino acid residues are inserted into, deleted from and/or substituted into the amino acid sequence relative to another polypeptide reference sequence. Variants can include variants of fusion proteins.

The term “fusion protein” indicates that the protein includes polypeptide components derived from more than one parental protein or polypeptide. Typically, a fusion protein is expressed from a “fusion gene” in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein. Fusion proteins incorporating an antibody or an antigen-binding portion thereof are known.

The inventive automated TFF perfusion bioreactor is configured and useful for culturing eukaryotic cells, e.g., mammalian, insect, algal, or yeast cells. For example, eukaryotic recombinant host cells capable of producing a secreted protein of interest can be cultured in the inventive TFF perfusion bioreactor. A “secreted” protein refers to those proteins capable of being directed to the endoplasmic reticulum (ER), secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments, the antibody protein of interest can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion (i.e., “protein-secreting” cells, e.g., protein-secreting mammalian cells), or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing an ionic detergent or chaotropic agent, such as sodium dodecyl sulfate (SDS), urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc., or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing an ionic detergent or chaotropic agent, such as sodium dodecyl sulfate (SDS), urea, guanidinium hydrochloride, or lithium perchlorate.

The term “naturally occurring,” where it occurs in the specification in connection with biological materials such as polypeptides, nucleic acids, host cells, and the like, refers to materials which are found in nature.

The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well-known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques.

The term “recombinant protein” or “recombinant polypeptide” as used herein refers to a protein molecule, e.g., a protein of interest, which is expressed using a recombinant DNA molecule. A “recombinant host cell” is a cell that contains and/or expresses a recombinant nucleic acid.

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

A “promoter” is a region of DNA including a site at which RNA polymerase binds to initiate transcription of messenger RNA by one or more downstream structural genes. Promoters are located near the transcription start sites of genes, on the same strand and upstream on the DNA (towards the 5′ region of the sense strand). Promoters are typically about 100-1000 bp in length.

An “enhancer” is a short (50-1500 bp) region of DNA that can be bound with one or more activator proteins (transcription factors) to activate transcription of a gene.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoranilidate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single-stranded or double-stranded and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express the immunoglobulin (e.g., antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

An expression cassette is a typical feature of recombinant expression technology. The expression cassette includes a gene encoding a protein of interest, e.g., a gene encoding an antibody sequence, such as an immunoglobulin light chain and/or heavy chain sequence. A eukaryotic “expression cassette” refers to the part of an expression vector that enables production of protein in a eukaryotic cell, such as a mammalian cell. It includes a promoter, operable in a eukaryotic cell, for mRNA transcription, one or more gene(s) encoding protein(s) of interest and a mRNA termination and processing signal. An expression cassette can usefully include among the coding sequences, a gene useful as a selective marker. In the expression cassette promoter is operably linked 5′ to an open reading frame encoding an exogenous protein of interest; and a polyadenylation site is operably linked 3′ to the open reading frame. Other suitable control sequences can also be included as long as the expression cassette remains operable. The open reading frame can optionally include a coding sequence for more than one protein of interest.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Recombinant expression technology typically involves the use of a recombinant expression vector comprising an expression cassette and a mammalian host cell comprising the recombinant expression vector with the expression cassette or at least the expression cassette, which may for example, be integrated into the host cell genome.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (See, e.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1). For expression of multi-subunit proteins of interest, separate expression vectors in suitable numbers and proportions, each containing a coding sequence for each of the different subunit monomers, can be used to transform a host cell. In other embodiments, a single expression vector can be used to express the different subunits of the protein of interest.

The term “host cell” means a cell that has been transformed, or is capable of being transformed, with a nucleic acid and thereby expresses a gene or coding sequence of interest. The term includes the progeny of the parent cell, whether or not the progeny is identical in morphology or in genetic make-up to the original parent cell, so long as the gene of interest is present. Any of a large number of available and well-known host cells may be used in the practice of this invention to obtain antibody variants, although mammalian host cells capable of post-translationally glycosylating antibodies are preferred. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, biosafety and costs. A balance of these factors must be struck with the understanding that not all hosts may be equally effective for the expression of a particular DNA sequence. Modifications can be made at the DNA level, as well. The peptide-encoding DNA sequence may be changed to codons more compatible with the chosen host cell. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

Within these general guidelines, microbial host cells in culture, such as bacteria (such as Escherichia coli sp.), and eukaryotic yeast cell lines (e.g., species and strains of Saccharomyces, Pichia, Schizosaccharomyces, Kluyveromyces) and other fungal cells, algal cells (see, e.g., Specht et al., Micro-algae come of age as a platform for recombinant protein production, Biotechnol. Lett. 32:1373-1383 (2010)) or algal-like cells, insect cells (e.g., Drosophila spp.; see, e.g., Yee et al., The Coming Age of Insect Cells for Manufacturing and Development of Protein Therapeutics, Ind. Eng. Chem. Res. 57(31):10061-10070 (2018)), plant cells (e.g., Nicotinia spp.; see, e.g., Karki et al., Cellular engineering of plant cells for improved therapeutic protein production, Plant Cell Reports 40:1087-1099 (2021)), that have been modified to incorporate humanized glycosylation pathways, can also be used to produce fully functional glycosylated proteins, e.g., antibodies. However, mammalian (including human) host cells, e.g., CHO cells and HEK-293 cells, are particularly in common use.

Examples of mammalian host cell lines, useful in the context of the inventive automated TFF perfusion bioreactor, are Chinese hamster ovary cells, including CHO-K1 cells (e.g., ATCC CCL61), CHO-S, DXB-11, DG-44, and Chinese hamster ovary cells/-DHFR (CHO, Urlaub et al, Proc. Natl. Acad. Sci. USA 77: 4216 (1980)); monkey kidney CVI line transformed by SV40 (COS-7, ATCC CRL 1651); human embryonic kidney line (293 or 293 cells subcloned for growth in suspension culture (Graham et al, J. Gen Virol. 36: 59 (1977)); baby hamster kidney cells (BHK, ATCC CCL 10); mouse Sertoli cells (TM4, Mather, Biol. Reprod. 23: 243-251 (1980)); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68 (1982)); MRC 5 cells or FS4 cells; or mammalian myeloma cells, e.g., NS0 or sp2/0 mouse myeloma cells. Other eukaryotic cells that are useful in the context of the inventive automated TFF perfusion bioreactor include: yeast cells, such as, but not limited to, Saccharomyces cerevisiae (e.g., EBY100) or Pichia pastoris; algal cell lines, such as, but not limited to, Chlamydomonas reinhardtii, Closterium peracerosum, Gonium pectorale, Haematococcus pluvialis, Lotharella amoebiformis, Ulva pertusa, Cyanidioschyzon merolae, or Dunaliella salina; insect cell lines, such as, but not limited to, Drosophila Schneider S2 cells, Spodoptera frugiperda (Sf9 or Sf21) cells, Antheraea eucalypti cells; and plant cells, such as, but not limited to, tobacco BY-2 cells.

“Cell,” “cell line,” and “cell culture” are often used interchangeably and all such designations herein include cellular progeny. For example, a cell “derived” from a CHO cell is a cellular progeny of a Chinese Hamster Ovary cell, which may be removed from the original primary cell parent by any number of generations, and which can also include a transformant progeny cell. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Host cells are transformed or transfected with the above-described nucleic acids or vectors for production of polypeptides (including antigen binding proteins, such as antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The invention is useful for culturing eukaryotic cells (e.g., mammalian cells) in one or more reusable or single-use perfusion bioreactors comprising a liquid culture medium under conditions that allow the cells to secrete the protein of interest into the medium for a production cultivation period of at least 10 days, and preferably, about 13-90 days, for example, about 20 to about 60 days.

Mammalian cells, such as CHO and BHK cells, are generally cultured as suspension cultures. That is to say, the cells are suspended in a liquid cell culture medium, rather than adhering to a solid support. Another useful mode of production is a hollow fiber bioreactor with an adherent cell line. Porous microcarriers can be suitable and are available commercially, sold under brands, such as Cytoline®, Cytopore®, or Cytodex® (GE Healthcare Biosciences).

A “cell culture” means the extracellular culture medium (fresh or conditioned) and the eukaryotic (e.g., mammalian, insect, algal, or yeast) cells cultured therein.

“Cell culture medium” or “culture medium,” used interchangeably herein, is a sterile aqueous medium suitable for growth of cells, and preferably eukaryotic cells (e.g., animal cells, such as insect or mammalian cells, e.g., CHO cells), in in vitro cell culture. “Feed medium” is fresh cell culture medium added to a cell culture after inoculation of the cells into the cell culture medium and cell growth has been commenced.

The term “production cultivation period” means the period during which protein-secreting mammalian cells are kept under incubation conditions in the bioreactor(s) which physiologically permit the continued production of the protein of interest. In some embodiments, expression of the protein can be constitutive; in other embodiments, expression of the protein can be engineered to be inducible (e.g., TetO-regulated expression). With such inducible expression, the production cultivation period includes only the period of cultivation in the bioreactor(s) when the inducer molecule (e.g., tetracycline, doxycycline, or other tetracycline analog) is present in the culture medium in sufficient quantities to induce expression of the protein of interest. For purposes of the invention, the production cultivation period is at least 10-90 days, or more, or at least 20 days, or more, e.g., 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or more; or 10-20 days, or more, or 20-30 days, or more, or 30-45 days, or more, or 45-60 days, or more, or 60-75 days, or 80-90 days, or more.

During the production cultivation period, fresh sterile liquid culture medium is periodically added into the one or more perfusion bioreactors. As may be desired, the fresh culture medium can be mixed in advance, or contemporaneously, from a plurality of different concentrated medium component solutions and an aqueous diluent. The phrase “mixed contemporaneously” means that the concentrated medium components and diluent are mixed together to make fresh culture medium, only within a few seconds or minutes (≤2 minutes) of when needed to replace volumes of medium that are removed from each of the perfusion bioreactor(s), either as volumes of permeate or cell bleed. A bioreactor has a characteristic mixing time, based on bioreactor and impeller design, and the agitation rate. For example, the Xcellerex® XDR 500-L SUB has blend time(s) from 30-55 seconds at agitation rates of 95-150 rpm. Shorter blend times are also possible by increasing agitation.

A “permeate” is a volume of conditioned cell culture medium fluid which has been filtered by microfiltration to remove all cells and contains the protein of interest. The conditioned medium upstream of the cell-removing microfilter(s), is called the “retentate,” and the conditioned medium downstream of the microfilter(s) is the “permeate,” which emerges from the perfusion system of the perfusion bioreactor and is ready for further processing. The flow rate of retentate cell culture fluid through the lumens of the perfusion filter, within the recirculation loop between the first port and the second port of the bioreactor, is called the “crossflow.” The flow of permeate cell culture fluid across the membrane of the perfusion filter is called the “flux.” The flux is typically measured as the volume of filtrate (permeate) that flows across the membrane of a perfusion filter per unit area of filtration surface within the filter per unit time of perfusion operation. As the permeate flux exits the perfusion system it is collected in a “permeate receptacle,” which generally can be any component or sequential unit operation that directly receives the permeate from the automated TFF perfusion bioreactor. For example, the “permeate receptacle” can be an additional filtration unit operation, such as depth filtration (e.g., in a filter bank), or a heat exchanger, or a first chromatography system (e.g., Protein A affinity chromatography system), or a container, such as, but not limited to, a surge vessel, a bag, barrel, or a carboy, or a drain, which collects the permeate flowing out of the perfusion microfilter. The permeate receptacle can be formatted for a single use, or it can be a receptacle formatted for re-use.

The term “sieving” means the product retention ratio across the perfusion filter, which is typically calculated as:

$$Sieving = \frac{Measured product titer in the bioreactor}{Measured product titer in the permeate} \times 100 \% .$$

“Transmembrane pressure” (TMP) means the hydrostatic pressure differential between the two sides of a membrane. In Tangential Flow Filtration (TFF) operation, this can be calculated as:

$$T M P = \frac{P 1 + P 2}{2} - P 3 ,$$

where P1 is the inlet pressure (entering the lumen), P2 is the retentate pressure (exiting the lumen), and P3 is the permeate pressure (exiting the membrane through the extracapillary space).

A “cell bleed” is a volume of cell culture, including some cells and culture medium, which is voided from the bioreactor(s) to waste and/or for analysis. The fresh culture medium is added to the bioreactor(s) periodically or continuously, depending on whether the removal of volumes of cell culture from the bioreactor(s) occurs intermittently (i.e., “periodically”) or continuously.

The term “buffer” or “buffered solution” refers to solutions which resist changes in pH by the action of their conjugate acid-base range. Examples of useful buffers include acetate, MES, citrate, Tris, bis-tris, histidine, arginine, succinate, citrate, glutamate, and lactate, or a combination of two or more of these, or other mineral acid or organic acid buffers; phosphate is another example of a useful buffer. Salts containing sodium, ammonium, and potassium cations are often used in making a buffered solution.

A “domain” or “region” (used interchangeably herein) of a polynucleotide is any portion of the entire polynucleotide, up to and including the complete polynucleotide, but typically comprising less than the complete polynucleotide. A domain can, but need not, fold independently (e.g., DNA hairpin folding) of the rest of the polynucleotide chain and/or be correlated with a particular biological, biochemical, or structural function or location, such as a coding region (e.g., encoding a protein of interest) or a regulatory region.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

Quantification of a protein of interest, is often useful or necessary to track production by the eukaryotic cells cultured in the inventive automated TFF perfusion bioreactor, and the yield, or appropriately formulate the protein (or a protein drug substance from a downstream unit operation) for further processing or storage. An antibody that specifically binds a domain of the protein of interest, particularly a specific monoclonal antibody, can therefore be useful for these purposes. In addition, the protein of interest that is being produced, isolated and purified during the production cultivation period, can be an antigen-binding protein, immunoglobulin, antibody, or antibody fragment, of any of the varieties described herein.

The term “antibody”, or interchangeably “Ab”, is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)2, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies that are antigen binding proteins are highly specific binders, being directed against an individual antigenic site or epitope, in contrast to polyclonal antibody preparations that typically include different antibodies directed against different epitopes. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab, Fab′, F(ab)₂, Fv, single chain antibodies, diabodies), maxibodies, nanobodies, and recombinant peptides comprising CDRs of the foregoing as long as they exhibit the desired biological activity, or variants or derivatives thereof.

The modifier “monoclonal” indicates the character of the antibody as being obtained from a substantially homogeneous population of antibodies, and is not to be construed as requiring production of the antibody by any particular method. For example, monoclonal antibodies may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The term “immunoglobulin” encompasses full or partial antibodies comprising two dimerized heavy chains (HC), each covalently linked to a light chain (LC); a single undimerized immunoglobulin heavy chain and covalently linked light chain (HC+LC), or a chimeric immunoglobulin (light chain+heavy chain)-Fc heterotrimer (a so-called “hemibody”), or a fusion protein comprising a dimerized or undimerized Fc domain, e.g., a peptibody. An “immunoglobulin” is a protein, but is not necessarily an antigen binding protein, e.g., a carrier antibody which is covalently linked to a clinically relevant target-binding moiety. On the other hand, an immunoglobulin can be designed to be bispecific or polyspecific binders of multiple clinically relevant targets. The term “peptibody” refers to a fusion protein molecule comprising an antibody Fc domain (i.e., at least the C_{H}2 and C_{H}3 antibody domains) that excludes antibody C_{H}1, CL, VH, and VL domains as well as Fab and F(ab)₂, wherein the Fc domain is attached to one or more peptides, preferably a pharmacologically active peptide. The production of peptibodies is generally described in PCT publication WO00/24782.

In an “antibody”, each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies. The constant region is the same among different antibodies. Within the variable region of each heavy or light chain, there are three hypervariable subregions that help determine the antibody's specificity for antigen in the case of an antibody that is an antigen binding protein. The variable domain residues between the hypervariable regions are called the framework residues and generally are somewhat homologous among different antibodies. Immunoglobulins can be assigned to different classes depending on the amino acid sequence of the constant domain of their heavy chains. Human light chains are classified as kappa (.kappa.) and lambda (.lamda.) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)). An “antibody” also encompasses a recombinantly made antibody, and antibodies that are glycosylated or lacking glycosylation.

The term “light chain” or “immunoglobulin light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” or “immunoglobulin heavy chain” includes a full-length heavy chain and fragments thereof having sufficient variable region sequence to confer binding specificity. A full-length heavy chain includes a variable region domain, V_{H}, and three constant region domains, C_{H1}, C_{H2}, and C_{H3}. The V_{H} domain is at the amino-terminus of the polypeptide, and the C_{H} domains are at the carboxyl-terminus, with the C_{H3} being closest to the carboxy-terminus of the polypeptide. Heavy chains are classified as mu (u), delta (8), gamma (γ), alpha (α), and epsilon (ε) and define the antibody's isotype as IgM, IgD, IgG, IgA, and IgE, respectively. Heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (Fc.gamma.Rs) on the surface of immune effector cells such as natural killers and macrophages, leading to the phagocytosis or lysis of the targeted cells. In CDC, the antibodies kill the targeted cells by triggering the complement cascade at the cell surface. An “Fc region”, or used interchangeably herein, “Fc domain” or

“immunoglobulin Fc domain”, contains two heavy chain fragments, which in a full antibody comprise the C_{H1} and C_{H2} domains of the antibody. The two heavy chain fragments are held together by two or more disulfide bonds and by hydrophobic interactions of the C_{H3} domains.

The term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and JH segments and may extend over as many as 10 nucleotides. Furthermore, several nucleotides may be inserted between the D and JH and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR (i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, th Ed. Public Health Service, National Institutes of Health, Bethesda, Md. (1991)). Even a single CDR may recognize and bind antigen, although with a lower affinity than the entire antigen binding site containing all of the CDRs.

An alternative definition of residues from a hypervariable “loop” is described by Chothia et al., J. Mol. Biol. 196: 901-917 (1987) as residues 26-32 (L1), 50-52 (L2) and 91-96 (L3) in the light chain variable domain and 26-32 (H1), 53-55 (H2) and 96-101 (H3) in the heavy chain variable domain.

“Framework” or “FR” residues are those variable region residues other than the hypervariable region residues.

The protein of interest can also be or include one or more antibody fragments. “Antibody fragments” comprise a portion of an intact full-length antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment which contains the constant region. The Fab fragment contains all of the variable domain, as well as the constant domain of the light chain and the first constant domain (CH1) of the heavy chain. The Fc fragment displays carbohydrates and is responsible for many antibody effector functions (such as binding complement and cell receptors), that distinguish one class of antibody from another.

Pepsin treatment yields an F(ab′)₂ fragment that has two “Single-chain Fv” or “scFv” antibody fragments comprising the V_{H} and V_{L} domains of antibody, wherein these domains are present in a single polypeptide chain. Fab fragments differ from Fab′ fragments by the inclusion of a few additional residues at the carboxy terminus of the heavy chain CH1 domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 1 13, Rosenburg and Moore eds., Springer-Verlag, New York, pp. 269-315 (1994).

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and a portion of one heavy chain that contains the V_{H} domain and the C_{H1} domain and also the region between the C_{H1} and C_{H2} domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference in their entireties.

“Single-chain Fv” or “scFv” antibody fragments comprise the V_{H} and V_{L} domains of antibody, wherein these domains are present in a single polypeptide chain, and optionally comprising a polypeptide linker between the V_{H} and V_{L} domains that enables the Fv to form the desired structure for antigen binding (Bird et al., Science 242:423-426, 1988, and Huston et al., Proc. Nati. Acad. Sci. USA 85:5879-5883, 1988). An “Fd” fragment consists of the V_{H} and C_{H1} domains.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The term “antigen binding protein” (ABP) includes antibodies or antibody fragments, as defined herein, that specifically bind a target ligand or antigen of interest.

In general, an antigen binding protein, e.g., a protein of interest, such as an immunoglobulin protein, or an antibody or antibody fragment, “specifically binds” to a target ligand or antigen of interest when it has a significantly higher binding affinity for, and consequently is capable of distinguishing, that target ligand or antigen, compared to its affinity for other unrelated proteins, under similar binding assay conditions. Typically, an antigen binding protein is said to “specifically bind” its target antigen when the dissociation constant (K_{D}) is 10⁻⁸ M or lower. The antigen binding protein specifically binds antigen with “high affinity” when the K_{D} is 10⁻⁹ M or lower, and with “very high affinity” when the K_{D} is 10⁻¹⁰ M or lower.

“Antigen binding region” or “antigen binding site” means a portion of a protein that specifically binds a specified target ligand or antigen. For example, that portion of an antigen binding protein that contains the amino acid residues that interact with a target ligand or an antigen and confer on the antigen binding protein its specificity and affinity for the antigen is referred to as “antigen binding region.” In an antibody, an antigen binding region typically includes one or more “complementary binding regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen. In a traditional antibody, the CDRs are embedded within a framework in the heavy and light chain variable region where they constitute the regions responsible for antigen binding and recognition. A variable region of an immunoglobulin antigen binding protein comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra).

The term “target” or “antigen” refers to a molecule or a portion of a molecule capable of being bound by a selective binding agent, such as an antigen binding protein (including, e.g., an antibody or immunologically functional fragment of an antibody), and additionally capable of being used in an animal to produce antibodies capable of binding to that antigen. An antigen may possess one or more epitopes that are capable of interacting with different antigen binding proteins, e.g., with antibodies.

The term “epitope” is the portion of a target molecule that is bound by an antigen binding protein (for example, an antibody or antibody fragment). The term includes any determinant capable of specifically binding to an antigen binding protein, such as an antibody or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three-dimensional structure that is similar to an epitope used to generate the antigen binding protein, yet comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three-dimensional structural characteristics, and/or specific charge characteristics. Generally, antigen binding proteins specific for a particular target will preferentially recognize an epitope on the target in a complex mixture of proteins and/or macromolecules.

The term “modification” when used in connection with proteins of interest, includes, but is not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. By methods known to the skilled artisan, proteins, can be “engineered” or modified for improved target affinity, selectivity, stability, and/or manufacturability before the coding sequence of the “engineered” protein is included in the expression cassette.

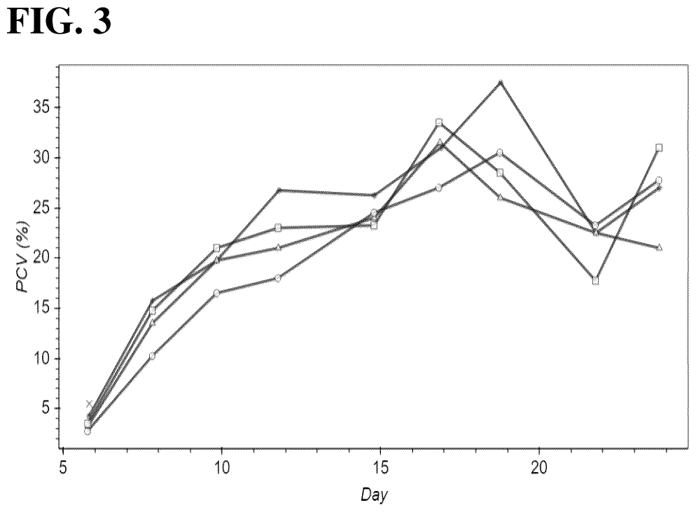

The term “surge vessel” means a storage reservoir, mixing vessel, feed tank, or collection vessel (or interchangeably, a “collection tank”), at the downstream end of a conduit, feeder, dam, pipe, or tubing, to absorb discrepant flow rates between two fluidly connected unit operations, e.g., the flow rate of a permeate coming from a bioreactor and the flow rate of a first chromatography system under automated control in continuous or semi-continuous format process embodiments of the invention. The surge vessel absorbs changes or differences in flow rates by allowing the volume to surge within pre-set volume range limits between the fluidly connected unit operations (see, e.g., FIG. 3). For purposes of the invention, surges vessels typically contain up to 50-650 L in volume; in semi-continuous process embodiments, 100-L to 650-L vessels are most useful, while in continuous process embodiments, 50-L to 200-L vessels are usually sufficient. In some embodiments, unit operations downstream of TFF perfusion bioreactor can optionally involve batch-wise processing of the product pool (e.g., virally inactivated and/or filtered by depth filtration to yield a filtered virally inactivated product pool (FVIP)); in such embodiments, the product pool can be collected in a collection vessel, and in subsequent batch-wise steps or operations, the further purified or processed product pool or filtrate can optionally be collected in other collection vessels between steps. In such discrete operation, batch-wise, or batch mode, processing, the collection vessel(s) or interchangeably “collection tank(s),” from one step (which in certain embodiments may also be deemed a “feed tank(s)” for the subsequent step) lack the automated controls of a surge vessel, and although the collection vessel (or feed tank) may physically resemble a surge vessel, for purposes of the invention such a collection vessel (or interchangeably, “collection tank”) or feed tank, is called a “holding vessel” or, interchangeably an “HV” (e.g., HV1, HV2, HV3, HV4, or HV5). A “holding vessel” can be a single-use holding vessel (SUHV), distinct from a single-use collection vessel (SUCV, e.g., SUCV1 or SUCV2) in a continuous or semi-continuous format set of manufacturing process steps or operations.

A “chromatography system” is an arrangement of at least one enclosed chromatography matrix, with closed conduit hardware (e.g., pipes or tubing) for fluid ingress and egress from the at least one chromatography matrix. The chromatography system involves one or more pumps and/or valves to automatically or manually control the fluid flow rate and pressure. The first, second and third (or additional) chromatography systems of a biologics manufacturing process and facility can incorporate chromatography matrices of various sorts, which the skilled practitioner knows how to select and use in sequence, as appropriate for the protein of interest. Encompassed within the term “matrix” are resins, beads, nanoparticles, nanofibers, hydrogels, membranes (e.g., membrane adsorbers (MAs)), and monoliths, or any other physical matrix, bearing a relevant covalently bound chromatographic ligand (e.g., Protein A, Protein G, or other affinity chromatographic ligand, such as a target ligand, a charged moiety, or a hydrophobic moiety, etc.). The matrix to which the affinity target ligand is attached is typically agarose, but other matrices are available. For example, mechanically stable matrices such as controlled pore glass, methacrylate (e.g., in Amsphere™ A3 resin; JRS Life Sciences), or poly(styrenedivinyl)benzene allow for greater stability, faster flow rates and shorter processing times than can be achieved with agarose. Where the protein comprises a C_{H3} immunoglobulin domain, the Bakerbond ABX™ resin (J. T. Baker, Phillipsburg, N.J.) can be useful for purification. An affinity chromatography matrix may be placed or packed into a column useful for the purification of proteins. Loading of a cell-free cell culture fraction onto an affinity chromatography matrix, e.g., in the first chromatography system, typically occurs at about neutral pH.

The term “to bind” or “binding” a molecule to Protein A, or a Protein A matrix, or another (different) affinity chromatography matrix, means exposing the molecule to the affinity chromatography ligand covalently bound to a solid substrate (e.g., a resin), under appropriate conditions (e.g., pH and selected salt/buffer composition), such that the molecule of interest is reversibly immobilized in, or on, the affinity chromatography ligand by virtue of its binding affinity under those conditions, regardless of the physical mechanism of affinity that may be involved. (See, e.g., Jendeberg, L. et al., The Mechanism of Binding Staphylococcal Protein A to Immunoglobin G Does Not Involve Helix Unwinding, Biochemistry 35(1): 22-31 (1996); Nelson, J. T. et al., Mechanism of Immobilized Protein A Binding to Immunoglobulin G on Nanosensor Array Surfaces, Anal. Chem., 87(16):8186-8193 (2015)).

The term “to bind” or “binding” a molecule to an ion exchange matrix (e.g., a CEX matrix, such as a CEX resin or membrane adsorber, or an AEX matrix, such as an AEX resin or membrane adsorber), means exposing the molecule to the ion exchange matrix under appropriate conditions (e.g., pH and selected salt/buffer composition) such that the molecule is reversibly immobilized in, or on, the ion exchange matrix by virtue of ionic interactions between the molecule and a charged group or charged groups (i.e., charged ligands) of the ion exchange matrix.

The term “loading buffer” or “equilibrium buffer” refers to the buffer, and salt or salts, which is mixed with a protein preparation (e.g., a batch or perfusion cell culture permeate or filtrate, or an eluant pool containing the protein of interest) for loading the protein preparation onto a Protein A matrix or other affinity chromatography matrix, or onto an ion exchange matrix (e.g., a CEX matrix or AEX matrix), or onto a hydrophobic interaction chromatography (HIC) matrix, as the case may be. This buffer is also used to equilibrate the chromatography matrix before loading, and to wash after loading the protein.

The term “wash buffer” is used herein to refer to the buffer that is passed over a Protein A matrix or another affinity chromatography matrix, or ion exchange matrix (e.g., a CEX matrix or AEX matrix), or a hydrophobic interaction chromatography (HIC) matrix, as the case may be, following loading of a protein preparation and prior to elution or after flow-through of the protein of interest. The wash buffer may serve to remove one or more contaminants without substantial elution of the desired protein or can be used to wash out a non-binding protein.

The term “elution buffer” or “eluant” refers to the buffer used to elute the protein of interest reversibly bound to a matrix. As used herein, the term “solution” refers to either a buffered or a non-buffered solution, including water.

The term “elution pool” or “eluant pool” means the material eluted from a matrix, which material includes the recombinant protein of interest.

The term “loading,” with respect to a Protein A matrix or other affinity chromatography matrix, or an ion exchange matrix (e.g., a CEX matrix), or a hydrophobic interaction chromatography (HIC) matrix, means loading a protein preparation (e.g., a batch or perfusion cell culture permeate or filtrate, or an eluant pool containing the protein of interest) onto the Protein A matrix or another affinity chromatography matrix, or the ion exchange matrix, or the HIC matrix.

The term “washing,” with respect to a Protein A matrix or other affinity chromatography matrix, or an ion exchange matrix (e.g., a CEX matrix or AEX matrix), or a HIC matrix, means passing an appropriate buffer through or over the Protein A matrix or ion exchange matrix or HIC matrix or other chromatographic matrix, as the case may be.

The term “eluting” a molecule (e.g., a desired recombinant protein or contaminant) from a Protein A matrix or another affinity chromatography matrix, or an ion exchange matrix (e.g., a CEX matrix or AEX matrix), or an HIC matrix, means removing the molecule from such material, typically by passing an elution buffer over the chromatography matrix.

The terms “single-use” or “single use” component(s), used interchangeably, means that a particular aseptic production line component, i.e., an aseptic piece of equipment, used in the inventive automated facility or in performing the inventive process is constructed or configured to be employed for a single production run (but may be re-used if quality and aseptic sanitation can be assured for multiple runs). The single-use component can then be disposed of and replaced for subsequent production runs by a another single-use component of the same or modified configuration without the need for cleaning and sanitization of the component between production runs. Examples of single-use components that can be employed in the production process for the protein of interest include, but are not limited to, the inventive automated TFF perfusion bioreactor and/or the recirculation loop, or any of the downstream unit operations, such as, but not limited to, a first chromatography system, a second chromatography system, a third chromatography system, a low pH or detergent viral inactivation system, a neutralization system, a viral filtration system, or an ultrafiltration/diafiltration system. Such single-use components can be constructed or obtained commercially, for example, but not limited to the following:

Single-use bioreactors: XCellerex® XDR single-use bioreactor bags (e.g., 500-L, 1000-L, or 2000-L volumes; GE Healthcare Life Sciences); BIOSTAT STR® stirred tank single-use bioreactor systems (e.g., 500-L to 2000-L volumes; Sartorius Stedim Biotech); HyPerforma Single-Use Bioreactors (e.g., 50-L, 100-L, 200-L, 500-L, 1000-L and 2000-L volumes; Thermo Fisher Scientific); Allegro™ Single-Use Stirred Tank Bioreactors (e.g., 500-L to 2000-L volumes; Pall); Millipore Mobius® Single-use Bioreactors (e.g., 500-L to 2000-L volumes; MilliporeSigma)) 50-L Rocking Bioreactor bags, including, but not limited to, Wave Bioreactor® Bag (GE Healthcare Life Sciences) or RIM Bio Rocker Bags; or mixer bags sold commercially by Pall or Sartorius (e.g., 100-L, 200-L, 650-L, 1000-L or 2000-L volumes);

Single-use perfusion systems: Spectrum Krosflo® Hollow Fiber Systems;

Single-use heat exchangers: Thermo Scientific™ DHX™ Heat Exchanger with a Thermo Scientific™ ThermoFlex™ Recirculating Chiller, and Thermo Scientific™ DHX™ Bag Assembly;

Single-use filter assembly systems containing filters (various membrane and pore sizes from MilliporeSigma or Sartorius Stedim Biotech), silicone and/or c-flex tubing, and aseptic connectors (from Pall, Colder, GE Healthcare Life Sciences, Sartorius Stedim Biotech);

Single-use transfer lines of various dimensions, lengths, and configurations using disposable aseptic connectors, silicone and/or c-flex type tubing are commercially available from Thermo Fisher Scientific (ASI) or Advantapure;

Single-use medium component solution or aqueous diluent (e.g., buffer) solution tote storage bags are sold commercially by Advanced Scientifics, Inc. (ASI; Thermo Fisher Scientific), MilliporeSigma, Sartorius, or RIM Bio;

Single-use viral inactivation systems: Cadence® Virus Inactivation System manifolds (Pall Life Sciences), FlexAct® for low pH Virus Inactivation (“VI”; Sartorius); Single-use chromatography systems: Cadence™ BioSMB® PD (Pall Life Sciences); Allegro™ Single Use Chromatography (Pall Biotech); Mobius® FlexReady Chromatography (MilliporeSigma); ÄKTA™ Ready Single Use Chromatography (GE Healthcare Life Sciences); or Sartobind® IEX membrane adsorbers (Sartorius Stedim Biotech);

Single-use viral filtration systems: Allegro™ MVP Single Use System Manifolds (Pall Biotech); Mobius® FlexReady for Viral Filtration (MilliporeSigma); FlexAct® for Viral Filtration (Sartorius), Planova™ Single-Use Virus Filtration (SU-VFS; Asahi Kasei Bioprocess America, Inc.), or Viresolve® Pro Virus Filtration (MilliporeSigma);

Single-use UF/DF systems: Allegro™ Single Use Tangential Flow Filtration System (Pall Biotech); Mobius® FlexReady TFF System (MilliporeSigma); FlexAct® for UF/DF (Sartorius); ÄKTA™ Readyflux single use filtration (GE Healthcare Life Sciences); and

Single-use aseptic connectors: AseptiQuik® connectors (Colder Products Company), Kleenpak® Presto Sterile Connector (Pall Biotech); Lynx® ST Connector (MilliporeSigma).

Employing single use components lends efficiency, safety, and lowers ultimate cost of biologics production process. Additionally, in scenarios where multiple single-use TFF perfusion bioreactors are utilized in a facility for the production of a purified protein of interest (e.g., but not limited to, a purified protein drug substance), multiple operations performed with respect to each bioreactor can be performed concurrently. For example, while an ultrafiltration/diafiltration operation is taking place with respect to the virus-free filtrate produced from a first perfusion bioreactor, a chromatography operation can be performed with respect to a virally inactivated product pool produced by the viral inactivation system (and, if needed, the neutralization system) processing a protein isolate fraction received after processing by the first chromatography system of cell-free permeate derived from culturing in a second single-use perfusion bioreactor. In another example, while an ultrafiltration/diafiltration operation is taking place with respect to the virus-free filtrate ultimately produced by culturing in a first single-use perfusion bioreactor, a viral filtration operation can be performed with respect to a virally inactivated product pool ultimately produced by culturing in a second perfusion bioreactor. In additional embodiments, at least one chromatography process and/or viral filtration process performed on virus-free filtrate produced from a first perfusion bioreactor can take place during continuous chromatography capture or viral inactivation processes performed on cell-free permeate volumes produced by a second single-use bioreactor in accordance with the inventive process.

The term “filter bank” or “filter assembly system”, used interchangeably refers to an apparatus that includes multiple filter assemblies with each filter assembly including at least one filter. A filter included in a filter assembly can be a single-use filter and replaced after a period of time and/or after an amount of use. A filter bank can be a portable piece of equipment. For example, a filter bank can be disposed on a filtration cart that can be moved to various locations in an automated facility. The filters included in a filter bank can include a filtration system comprising a depth filter, a 0.2 micrometer filter, a membrane filter, a 20 nanometer (nm) filter, a viral filtration device, an ultrafiltration device, a diafiltration device, or combinations thereof. A filter bank can be configured such that while material is flowing through at least one filter of the filter bank, another filter of the filter bank remains unused. In various embodiments, a filter bank can be coupled to a diverter valve or other flow control device to control the flow of material to the filters included in the filter bank. The diverter valve or flow control device can be pneumatically controlled.

The foregoing are merely exemplary, and not an exhaustive list, of single-use systems and connectors that are available to the skilled practitioner of the present invention.

### Proteins of Interest

The protein of interest to be manufactured using the present invention can be any industrially or medically useful protein, such as, but not limited to, a pharmacologically active protein or peptide.

For example, the protein of interest can be a mimetic or agonist peptide. The terms “-mimetic peptide,” “peptide mimetic,” and “-agonist peptide” refer to a peptide or protein having biological activity comparable to a naturally occurring protein of interest. These terms further include peptides that indirectly mimic the activity of a naturally occurring peptide molecule, such as by potentiating the effects of the naturally occurring molecule.

The protein of interest can be an antagonist peptide or inhibitor peptide. The term “-antagonist peptide,” “peptide antagonist,” and “inhibitor peptide” refer to a peptide or protein that blocks or in some way interferes with the biological activity of a receptor of interest, or has biological activity comparable to a known antagonist or inhibitor of a receptor of interest (such as, but not limited to, an ion channel or a G-Protein Coupled Receptor (GPCR)).

Examples of pharmacologically active proteins that can be manufactured with the present invention include, but are not limited to, an IL-6 binding peptide, a CD3 binding protein, a CD19 binding protein, a CD20 binding protein, a CD22 binding protein, a HER2 binding protein, a HER3 binding protein, a vascular endothelial growth factor-A (VEGF-A) binding protein, a TNF-α binding protein, an EGFR binding protein, a RANK ligand binding protein, an IL-1α binding protein, an IL-1β binding protein, an IL-17A binding protein, an EPCAM (CD326) binding protein, a CGRP peptide antagonist, a bradykinin B1 receptor peptide antagonist, a toxin peptide, a placental growth factor (PIGF) binding protein, a hormone (e.g., insulin, human growth hormone, or thyroxine) a parathyroid hormone (PTH) agonist peptide, a parathyroid hormone (PTH) antagonist peptide, an ang-1 binding peptide, an ang-2 binding peptide, a myostatin binding peptide, an erythropoietin-mimetic (EPO-mimetic) peptide, a FGF21 peptide, a thrombopoietin-mimetic (TPO-mimetic) peptide (e.g., AMP2 or AMPS), a nerve growth factor (NGF) binding peptide, a B cell activating factor (BAFF) binding peptide, and a glucagon-like peptide (GLP)-1 or a peptide mimetic thereof or GLP-2 or a peptide mimetic thereof.

Protein and coding sequences for such proteins, some of which have already received regulatory approval, are well known in the art. However, the present invention can also be applied to the manufacture of drug substances yet to be innovated by methods of drug discovery, research and development, and clinical trials.

### Cloning DNA

Cloning of DNA for the expression of the protein(s) of interest is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the protein of interest.

One common source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

Sequencing of DNA is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of genes and cDNAs, one of skill will readily be able to determine, depending on the region sequenced. One source of gene sequence information is the National Center for Biotechnology Information, National Library of Medicine, National Institutes of Health, Bethesda, MD. Gene sequencing can also be done, for example, by standard methods or by so-called “Next-generation” sequencing of engineered DNA constructs prior to transfection. (See, e.g., Buermans, H. P. J., & den Dunnen, J. T., Next generation sequencing technology: Advances and applications, Biochimica et Biophysica Acta—Molecular Basis of Disease 1842(10): 1932-1941 (2014)).

Chemical synthesis of parts or the whole of a coding region containing codons reflecting desires protein changes can be cloned into an expression vector by either restriction digest and ligation of 5′ and 3′ ends of fragments or the entire open reading frame (ORF), containing nucleotide overhangs that are generated by restriction enzyme digestion and which are compatible to the destination vector. The fragments or inserts are typically ligated into the destination vector using a T4 ligase or another common enzyme. Other useful methods are similar to the above except that the cut site for the restriction enzyme is at location different from the recognition sequence. Alternatively, isothermal assembly (i.e., “Gibson Assembly”) can be employed, in which nucleotide overhangs are generated during synthesis of fragments or ORFs; digestion by exonucleases is employed. Alternatively, nucleotide overhangs can be ligated ex vivo by a ligase or polymerase or in vivo by intracellular processes.

Alternatively, homologous recombination can be employed, similar to isothermal assembly, except exonuclease activity of T4 DNA ligase can used on both insert and vector and ligation can be performed in vivo.

Another useful cloning method is the so-called “TOPO” method, in which a complete insert containing a 3′ adenosine overhang (generated by Taq polymerase) is present, and Topoisomerase I ligates the insert into a TOPO vector.

Another useful cloning method is degenerate or error-prone PCR exploiting degenerate primers and/or a thermally stable low-fidelity polymerase caused by the polymerase within certain reaction conditions. Fragments or inserts are then cloned into an expression vector.

The above are merely examples of known cloning techniques, and the skilled practitioner knows how to employ any other suitable cloning techniques.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the expressed protein by the recombinant host cells); an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

### Protein Expression

Manufacturing a purified protein of interest (e.g., but not limited to, a protein drug substance) typically involves culturing protein-secreting eukaryotic cells, for which the inventive automated TFF perfusion bioreactor is particularly advantageous.

Such cultured cells are typically made by recombinant DNA technology involving transient or stable transfection, e.g., the pooled plasmid constructs (expression vectors) from the cloning step can be transfected into a plurality of host cells (e.g., mammalian, e.g., HEK 293 or CHO, bacterial, insect, yeast cells) for expression using a cationic lipid, polyethylenimine, Lipofectamine™, or ExpiFectamine™, or electroporation. The skilled practitioner is aware of numerous suitable means for transfecting to achieve expression of recombinant antibodies. Alternatively, methods for stable genomic integration of expressions cassettes encoding the protein of interest can be employed to make a production cell line of protein-secreting mammalian cells. (See, e.g., Zhang, Crispr-Cas Systems and Methods for Altering Expression Of Gene Products, WO2014093661 A2; Frendewey et al., Methods and Compositions for the Targeted Modification of a Genome, U.S. Pat. No. 9,228,208 B2; Church et al., Multiplex Automated Genome Engineering, WO2008052101A2, U.S. Pat. No. 8,153,432 B2; Bradley et al., Methods Cells and Organisms, US2015/0079680 A1; Begemann et al., Compositions and Methods for Modifying Genomes, WO2017141173A2; Gill et al., Nucleic acid-guided nucleases, U.S. Pat. No. 9,982,279 B1; Minshull et al., Enhanced nucleic acid constructs for eukaryotic gene expression, U.S. Pat. No. 9,428,767B2, U.S. Pat. No. 9,580,697B2, U.S. Pat. No. 9,574,209B2; Minshull et al., DNA Vectors, Transposons And Transposases For Eukaryotic Genome Modification, U.S. Pat. No. 10,041,077B2).

Optionally, the transfectant or transformant cells will be provided with a recombinant expression cassette for a selectable marker, for example, but not limited to, one or more of the following: glutamine synthase, dihydrofolate reductase, puromycin-N acetyl transferase, blasticidin-S deaminase, hygromycin phosphotransferase, aminoglycoside phosphotransferase, nourseothricin N-acetyl transferase, or a protein that binds to zeocin.

The protein of interest is typically obtained by culturing the transfected or transformed host cells under physiological conditions allowing the cells to express recombinant proteins. Most conveniently, the expressed recombinant proteins are directly secreted into the extracellular culture medium (by employing appropriate secretory-directing signal peptides) and are harvested therefrom; otherwise, additional steps will be needed to isolate the expressed antibodies from a cell extract.

The desired scale of the recombinant expression will be dependent on the type of expression system and the desired quantity of protein production. Some expression systems such as ExpiCHO™ usually produce higher yields as compared to some earlier HEK293 technologies. A smaller scale ExpiCHO™ might then suffice as compared to an HEK293 system. Efficiency of transfection can also be a consideration in choosing an appropriate expression system. Electroporation can be a suitable method given its effectiveness, relative low cost and the fact that high-throughput during this step is not critical. Additionally, the ratio of immunoglobulin light chain to heavy chain can be varied during the co-transfection to improve expression of certain variants. The product yield for a given variant must be sufficient to survive numerous handling steps and produce a signal high enough to be detected by the chosen fluorescence detector.

In general, the transfected or transformed host cells are typically cultured by any conventional type of culture, such as batch, fed-batch, intensified fed-batch, or continuous. Suitable continuous cultures included repeated batch, chemostat, turbidostat or perfusion culture with product and cell retention or solely cell retention. However, for purposes of the invention, culturing is carried out in one or more single-use perfusion bioreactors, each of which can contain a volume of liquid culture medium of about 50 L to about 4000 L (e.g., 50 L, 60 L, 75 L, 100 L, 250 L, 500 L, 650 L, 750 L, 1000 L, 1250 L, 1500 L, 1750 L, 2000 L, 2250 L, 2500 L, 2750 L, 3000 L, 3250 L, 3500 L, 3750 L, or 4000 L), as desired. The number of single-use bioreactors employed to culture the cells is one, two, three, four, five, or six single-use perfusion bioreactors of the desired volume(s).

The host cells used to produce the protein of interest or “POI” (e.g., non-glycosylated or glycosylated proteins) in the invention can be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Patent Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source, such that the physiological conditions of the cell in, or on, the medium promote expression of the protein of interest by the host cell; any other necessary supplements may also be included at appropriate concentrations that would be known to those skilled in the art.

Desired culture conditions can be set in advance, i.e., “predetermined,” and modified at will, by the skilled artisan; such culture conditions include, but are not limited to, temperature (for mammalian cells, typically, but not necessarily, about 37°±1° C.), pH (typically, but not necessarily, the cell culture medium is maintained within the range of about pH 6.5-7.5), oxygenation, and the like. By “culturing at” or “maintaining at” a predetermined culture condition, it is meant that the process control systems are set at a particular value for that condition; in other words, the intended volume, target temperature, pH, oxygenation level, or the like, are maintained at predetermined setpoints for each parameter, within a narrow range (i.e., “narrow deadband”) most optimal for the cell line and protein product of interest. Clearly, there will be small variations of the temperature, pH, or other culture condition over time, and from location to location through the culture vessel (i.e., the bioreactor). (See, also, e.g., Oguchi et al., pH Condition in temperature shift cultivation enhances cell longevity and specific hMab productivity in CHO culture, Cytotechnology. 52(3): 199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub-physiological temperatures, Mol. Biotechnol. 39:69-77 (2008)).

For purposes of the present invention, the direction of flow of cell-containing conditioned medium through the recirculation loop is one such “culture condition,” under the automatic control of a controller. The period of time set for flow to occur in the first direction, before the low-shear pump (or centrifugal pump) is automatically switched to direct flow through the recirculation loop in the second direction, is a predetermined period. Similarly, the period of time set for flow to occur in the second direction, before the low-shear pump (or centrifugal pump) is automatically switched to direct flow through the recirculation loop in the first direction, is a predetermined period. Preferably, but not necessarily, these two predetermined periods are approximately the same, or alternatively, the period of flow in the second direction may be different from the period of flow in the first direction. This predetermined period ranges from about 20 minutes to about 24 hours, or about 25 minutes to about 12 hours, or about 30 minutes to about three (3) hours, or about 45 minutes to about two (2) hours, or about one (1) hour.

As noted above, the automated tangential flow filtration perfusion bioreactor of the present invention includes a controller. Such controllers, or digital control units, and sensory monitors are available commercially or can be constructed by the skilled artisan to monitor, maintain, and/or modify culture conditions, including the periodic switching of the pumping direction of flow of the cell-containing liquid culture medium through the recirculation loop. Alternative digital control units (DCU) control and monitor the cell culture process are available commercially, made by companies such as B. Braun, New Brunswick, Sartorius, or Thermo Fisher Scientific. Table 1A (below) lists some examples of digital control and sensory equipment that can be used to monitor cell culture conditions. Other on-line or off-line analyses can include off-gas measurements by mass spectrometry, in-depth determination of media composition (amino acids, vitamins, trace minerals) and expanded examination of cellular metabolites other than CO₂ and lactic acid.

TABLE 1
Examples of commercially available cell culture control and sensory equipment.

| Equipment | Description |
|---|---|
| Digital Control Unit | Vendor- Specific (examples include Applikon, Wonderware |
| or PLC Logic | (Aveva), DeltaV (Emerson), APACS (Siemens), Allen- |
| Controllers | Bradley (Rockwell), etc. |
| pH Probe | Hamilton EasyFerm Plus (potentiometric) |
| Dissolved Oxygen | Hamilton VisiFerm (optical) or Broadley James OxyProbe ® |
| Probe | (polarographic) |
| Gas flow controller | Solenoid-controlled gas flow consoles and/or mass flow |
| | controllers (MFCs); multiple vendors |
| Blood gas analyzer | Siemens RapidLab ® 248 or Siemens Rapidpoint ® 500 |
| Cell counter | Beckman Coulter Vi-Cell ® XR or BioProfile ® CDV (Nova |
| | Biomedical Corp.) |
| Glucose, lactate and | YSI 2700 SELECT ™ Biochemistry analyzer (YSI Life |
| metabolite analyzer | Sciences) or BioProfile ® Basic 2 (Nova Biomedical Corp.) |
| Osmometer | Advanced Instruments Model 2020 |

The culture medium can include a suitable amount of serum such as fetal bovine serum (FBS), or preferably, the host cells can be adapted for culture in serum-free medium. In some embodiments, the aqueous medium is liquid, such that the host cells are cultured in a cell suspension within the liquid medium. The host cells can be usefully grown in continuous (perfusion) cell culture systems, preferably that are designed for single-use.

In accordance with the invention, fresh culture medium is periodically mixed (in advance or contemporaneously) from a plurality of concentrated component solutions and an aqueous diluent, or from powdered raw ingredients and an aqueous diluent, or a combination thereof. Cell culture media are complex mixtures that contain a wide range of concentrations of each component as well as unique ratios of one component to another. The factor by which any cell culture medium formulation can be concentrated is limited by the solubility, stability, or filterability of its least soluble, least stable, or least filterable component. By dissolving components as chemically compatible subgroups, increased concentration factors can be achieved that would otherwise not be possible if all the components were dissolved together. For example, some components are more soluble at acidic pH while others are more soluble at alkaline pH. In this example, components that are soluble at acidic pH can be grouped together in one solution while components that are soluble at alkaline pH can be grouped together in another solution in such a way that when they are recombined, they make a complete medium. In addition to or instead of pH grouping to achieve higher concentrations, one can utilize other solvents such as alcohol or dimethyl sulfoxide (DMSO); or, one can create stock solutions of individual components that have specialized solubility or storage requirements that necessitate their exclusion from other components until they are added to the bioreactor. The exact grouping of compatible components and their and maximum concentration for any given cell culture medium formulation is easily determined by those skilled in the art.

The term “viable cell density” (VCD) means the total number of viable cells per unit volume of culture, which is typically measured using a cell counter with a viable cell dye, such as trypan blue, in contrast to the “total cell density” (which is the total number of live and dead cells per mL of culture). A viable cell density can be used in the bioreactor from about 1.0×10⁶ up to about 2×10⁸ cells/mL, for example, in the range of 1.0×10⁶ to 2.0×10⁷ cells/mL, or in the range of about 4×10⁷ cells/mL to about 5×10⁷ cells/mL, or in the range of about 1×10⁸ cells/mL to about 2×10⁸ cells/mL. The inventive automated TFF perfusion bioreactor is particularly suited to support viable cell densities of about 60×10⁶/mL, or about 80×10⁶/mL, up to about 300×10⁶/mL, over a production cultivation period of 13-90 days. It is known that increasing the concentration of cells toward the higher end of the preferred ranges can improve volumetric productivity, an advantage that can be achieved by using the automated tangential flow filtration perfusion bioreactor of the present invention, because sieving through the hollow fiber filter or microfilter typically decreases by less than about 10% over a production cultivation period of 20-60 days at a viable cell density of about 100-150×10⁶/mL. Nevertheless, ranges of cell density including any of the above point values as lower or higher ends of a range are envisaged. The desired scale of the recombinant expression and cell culture will be dependent on the type of expression system and quantities of drug substance desired.

For purposes of the claimed invention, upon culturing the transfected or transformed host cells, the recombinant polypeptide or protein is directly secreted into the medium. Harvesting the recombinant protein involves separating it from particulate matter that can include host cells, cell aggregates, and/or lysed cell fragments, into a cell-free fraction that is free of host cells and cellular debris, i.e., a cell-free “permeate.” Such cells and cellular debris are removed from the conditioned medium, for example, by centrifugation and/or microfiltration. For example, to make the permeate, one can employ hollow fiber membranes (pore size 0.2 μm) or a series of filtration steps such as depth filtration, which can be configured on a mobile, interchangeable and/or single use and “filtration cart.”

Some embodiments of the invention include a first single-use surge vessel (SUSV1) as the permeate receptacle, adapted to receive volumes of permeate removed from the automated TFF perfusion bioreactor(s); the volumes of permeate are cell free. These permeate volumes are automatically and fluidly fed from the one or more single-use perfusion bioreactor(s) into the SUV1. In some embodiments, there is an automated controller comprising detectors to measure the fluid volume in SUSV1, and a processor to vary the pump speeds of the first chromatography system to maintain a pre-set volume range in the SUSV1.

In some embodiments of the invention, the facility for practicing the process further comprises a hollow fiber membrane, a series of depth filters, or a filtration cart, to make the permeate cell free before it is automatically and fluidly fed to the SUSV1.

### Protein Purification and Viral Inactivation

In general, the purification of proteins (e.g., recombinant or naturally occurring proteins) is usually accomplished by an optional series of chromatographic steps such as anion exchange chromatography, cation exchange chromatography, affinity chromatography (using Protein A or Protein G or Protein L as an affinity ligand or another different affinity ligand), hydrophobic interaction chromatography (HIC), hydroxy apatite chromatography, Reverse Phase HPLC, and size exclusion chromatography. The preceding are non-limiting examples of chromatographic modalities that can be included in any of the first chromatography system, the second chromatography system, and/or the third chromatography system. Any one of these chromatographic modalities can be selected as the permeate receptacle, adapted to receive volumes of permeate removed from the automated TFF perfusion bioreactor(s), if consistent with effective purification of a particular protein of interest.

Each of the first, second, or third chromatography system(s) can be configured as needed for the protein of interest, preferably with one, two, three or more different chromatographic matrices (e.g., chromatography columns) fluidly linked in succession, and which, optionally, can be arranged in a mobile, interchangeable, or disposable, single-use unit, skid or “cart.” Further, the purification process chosen may comprise one or more ultra-, nano- or diafiltration steps. Typically in a protein manufacturing process, the first, second, and/or optional third chromatography system(s) are configured as needed for the protein of interest, preferably with one, two, three or more different chromatographic matrices (e.g., chromatography columns) fluidly linked in succession, and which, optionally, can be arranged in a mobile, interchangeable, or disposable, single-use unit, skid or “cart.”

Other optional known techniques for protein purification such as ethanol precipitation, chromatofocusing, SDS-PAGE, and ammonium sulfate precipitation are also possible depending on the protein to be recovered, and any of these operations can optionally constitute the permeate receptacle), if consistent with effective purification of a particular protein of interest.

In some embodiments, the inventive automated TFF perfusion bioreactor is part of a process for manufacturing a purified protein of interest (e.g., a protein drug substance). The protein of interest (e.g., but not limited to, a protein drug) in the cell-free permeate is captured by one or more chromatographic capture steps of a first chromatography system, in the role of permeate receptacle adapted to receive volumes of permeate removed from the automated TFF perfusion bioreactor(s); the first chromatography system can partially purify and/or concentrate the protein, such as, but not limited to, Protein A or Protein G or Protein L affinity chromatography, or affinity chromatography employing a different affinity ligand covalently bound to a solid matrix. (See, e.g., Frank, M. B., “Antibody Binding to Protein A and Protein G beads” 5. In: Frank, M. B., ed., Molecular Biology Protocols. Oklahoma City (1997)). In some of these embodiments, the first chromatography system can optionally include anion exchange chromatography (AEX), cation exchange chromatography (CEX), affinity chromatography (using Protein A or Protein G or Protein L as an affinity ligand or another particular target moiety), hydrophobic interaction chromatography (HIC), hydroxy apatite (HA) chromatography and size exclusion chromatography (SEC). In some embodiments involving a surge vessel upstream, with the surge vessel serving as the permeate receptacle, which is fluidly connected to the first chromatography system, e.g., a first single-use surge vessel (SUSV1), there is an automated controller comprising detectors to measure the fluid volume in the surge vessel, e.g., the SUSV1, and a processor to vary the pump speeds of the first chromatography system to maintain a pre-set volume range in the surge vessel, (e.g., SUSV1). The volume of the SUSV1 is typically about 200 L, but can be set smaller or larger depending on the flow rates of the process and the desired residence time (which impacts the time frame allowed to react to process upsets). The operation of the first chromatography system collects or captures the protein of interest in a protein isolate fraction.

In some embodiments, the downstream second chromatography system comprises a single-use membrane adsorber (MA), such as, a surface-functionalized membrane. Such membrane adsorbers can involve anion-exchange groups for mAb polishing operations in negative mode, in which trace impurities are removed without binding the protein of interest (so-called “flow-through chromatography”). Examples, include, but are not limited to, Sartobind® Q or Sartobind STIC® (Sartorius Stedim Biotech), or Mustang® Q (Pall Life Sciences), or NatriFlo® HD-Q (Natrix Separations). Alternatively, membrane adsorbers can involve cation-exchange groups. e.g., Sartobind® s (Sartorius Stedim Biotech), or Mustang® S (Pall Life Sciences), or Natrix® HD-Sb (Natrix Separations). In some embodiments, membranes with other functional groups can be used to perform hydrophobic-interaction chromatography (HIC).

Purity of Water and other Ingredients. The water and all other ingredients that are used to express, purify and make formulations of proteins of interest, e.g., purified drug substances, are preferably of a level of purity meeting the applicable legal or pharmacopoeial standards required for such pharmaceutical compositions and medicaments in the jurisdiction of interest, e.g., United States Pharmacopeia (USP), European Pharmacopeia, Japanese Pharmacopeia, or Chinese Pharmacopeia, etc. For example, according to the USP, Water for Injection (“WFI”) is used as an excipient in the production of parenteral and other preparations where product endotoxin content must be controlled, and in other pharmaceutical applications, such as cleaning of certain equipment and parenteral product-contact components; and the minimum quality of source or feed water for the generation of Water for Injection is Drinking Water as defined by the U.S. Environmental Protection Agency (EPA), EU, Japan, or WHO.

### Automation and Control Systems

Conventional production facility control systems are typically designed to control a preset configuration of equipment. In these scenarios, the logical and hardware couplings between pieces of equipment do not change. Thus, the identifiers and control operations that can be performed with respect to each piece of equipment are static. The implementations of production facility control systems described herein, with respect to the inventive automated TFF perfusion bioreactor in the context of automated facilities and processes for manufacturing a purified protein of interest (e.g., but not limited to, a protein drug substance), support variable configurations of equipment in a production line. In these situations, a piece of equipment can have different functionality, perform different operations, and/or be controlled using different sets of control commands and/or variables based on the location of the piece of equipment within a production line. Thus, the production lines and control systems described herein include software configurations and physical hardware that are different from conventional systems. The ability to configure a production line within an automated facility using a same group of control modules with different arrangements of pieces of equipment on the production line can be an implementation of so-called “Flex Train” automation.

The implementations described herein can be performed by one or more systems that can automatically control the flow of material from the automated TFF perfusion bioreactor of the present invention, through each step of the downstream process to produce a purified protein of interest, such as but not limited to, a protein drug substance. Alternatively, at least a portion of the control functions can be performed by operator intervention, and there may be circumstances (especially process disruptions) that may require operator intervention. The control functions can be performed using process data obtained from sensors coupled to various pieces of equipment used in the production of the purified protein of interest. The sensors can include temperature sensors, pH sensors, flow rate sensors, weight sensors (e.g., load cells), volume sensors (e.g., guided wave radar sensors), pressure sensors, timers, capacitance sensors, optical density sensors, or combinations thereof. The data generated by the sensors can be collected locally by the pieces of equipment. In certain embodiments, the pieces of equipment can forward the sensor data to a production facility control system. The production facility control system can collect data from sensors of a number of pieces of equipment being used to manufacture the purified protein of interest (e.g., a purified protein drug substance). The production facility control system can include one or more computing devices and/or one or more data stores that are in electronic communication with each other. At least a portion of the one or more computing devices and/or one or more data stores can be located in a same location, in some scenarios. Additionally, at least a portion of the one or more computing devices and/or the one or more data stores can be located remotely from the equipment included in a production facility. In this situation, at least a portion of the operations performed by the production facility control system can be implemented in a cloud computing architecture.

The data collected from the sensors can be stored in electronic data stores that can be referred to herein as “data historians.” In various implementations, a first data historian can collect and store data for at least a subset of the pieces of equipment operating in the purified protein production facility (e.g., for the production of a purified protein drug substance or other protein of interest). The first historian can store data for a period of time and then forward the data to a second data historian that is a repository for data collected regarding the operation of pieces of equipment coupled to the production facility control system. DeltaV historian and/or Pi historian are examples of commonly used redundant data historian systems in a commercial manufacturing plant for protein drug substances. In certain situations, the first data historian can then be reset and begin collecting and storing additional data from the purified protein production facility (e.g., for production of a purified protein drug substance) for an additional period of time. The production facility control system can also include one or more batch historians that collect and store data related to the operation of pieces of equipment included in the production facility for the production of particular batches of the purified protein of interest (e.g., but not limited to, a protein drug substance). The data historians can be accessed by the production facility control system and analyzed to determine parameters for the operation of pieces of equipment included under the control of the production facility control system.

The production facility control system can analyze the data obtained from the sensors and determine operating conditions for one or more pieces of equipment, for example, the automated TFF perfusion bioreactor. In some cases, the setpoints and acceptable operating parameters, and/or run recipe for the operation of a piece of equipment, such as the automated TFF perfusion bioreactor, can be entered into the system by an operator. In other situations, the setpoints and acceptable operating parameters, and/or run recipe for the operation of a piece of equipment can be automatically sent to one or more pieces of equipment utilized in a purified protein production line (e.g., for the production of a purified protein drug substance). Alerts and alarm notifications can also be generated based on the sensor data. For example, in situations where sensor data indicates that an operating condition for a piece of equipment in a purified protein production line is outside of a threshold range, the system can trigger an alarm and send notification to an operator.

Various pieces of equipment, including the automated TFF perfusion bioreactor, used in a manufacturing process to produce a purified protein of interest (e.g., a purified protein drug substance), can include one or more communication interfaces that enable communications between the pieces of equipment and/or with the production facility control system. In some implementations the production facility control system can operate as a process automation system (PAS). The communication interfaces can include hardware devices, firmware devices, and/or software implemented systems that enable communication of data between pieces of equipment used in a purified protein production line and/or with the production facility control system. The communication interfaces can enable communication of data over a number of networks, such as local area wired networks, local area wireless networks, wide area wireless networks, and/or wide area wired networks. In particular examples, the communication interfaces can include Ethernet network communication interfaces, Internet Protocol network communication interfaces, Institute of Electrical and Electronics Engineers (IEEE) 802.11 wireless network communication interfaces, Bluetooth communication interfaces, or combinations thereof.

The automated TFF perfusion bioreactor of the invention, and the other pieces of equipment involved in the various unit operations used to produce the purified protein of interest (e.g., but not limited to, a protein drug substance), can include one or more processors and one or more memory devices. The one or more processors can be central processing units, such as standard programmable processors that perform arithmetic and logical operations necessary for the operation of computing systems. The one or more memory devices can include volatile and nonvolatile memory and/or removable and non-removable media implemented in any type of technology for storage of information, such as computer-readable instructions, data structures, program modules, or other data. Such computer-readable storage media can include, but is not limited to, RAM, ROM, EEPROM, flash memory or other memory technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, removable storage media, or any other medium that can be used to store the desired information and that can be accessed by the production facility control system or by the individual pieces of equipment included in a purified protein production line.

At least a portion of the pieces of equipment included in the purified protein production line, and the production facility control system can store one or more modules that can be executed to control the operation of the pieces of equipment included in the purified protein production line. For example, the controller of the automated TFF perfusion bioreactor can contain a module capable of automatically switching the low-shear pump system, directing the flow of the conditioned medium containing cells through the recirculation loop in the first direction, and after a predetermined period, automatically switching the low-shear pump system to direct the flow of the conditioned medium containing cells to the second direction, and vice versa. In this example, the module in the controller automatically switches the low-shear pump system to reverse the direction of flow through the recirculation loop periodically according to a pre-set schedule. Other modules of the controller may direct the periodic release of volumes of fresh cell culture medium (already mixed or in ratioed culture medium concentrate(s) form) from one or more medium reservoir(s) into the bioreactor's inlet for receiving volumes of fresh cell culture medium, in response to sensor data from the bioreactor measuring the contemporaneous culture conditions.

Such controller modules can include computer-readable instructions that can be executed to cause the other pieces of equipment included in the purified protein production line to take one or more actions. The modules can be part of a framework that enables the pieces of equipment included in the purified protein production line to produce the purified protein (e.g., the purified protein drug substance) in a continuous or semi-continuous manner. The actions performed by various pieces of equipment included in the purified protein production line can be related to start up processes, hold processes, shutdown processes, feed processes, or end of production processes.

In particular embodiments of the controller, the control systems described herein can be used to control production lines that have flexible configurations. That is, the control systems described herein can accommodate multiple configurations that utilize portable equipment that can be coupled to other components of the production line. In various embodiments, the production line can include one or more skids that include original manufacturer's equipment, such as a single-use bioreactor system, a perfusion system, or a continuous chromatography system. The skids can also include flow control devices, such as pumps. For example, in some embodiments of the automated TFF perfusion bioreactor, the perfusion system and/or the low-shear (e.g., centrifugal) pump system is located on a portable skid, with the recirculation loop fluidly connecting the bioreactor with the TFF perfusion system. Additionally, the skids can include one or more communication interfaces, also referred to herein as “drops,” that enable the physical coupling of portable pieces of equipment to the skid. The physical coupling between the portable pieces of equipment and the skid can be achieved using electrical cabling. The electrical cabling can be configured to enable ethernet communications. In certain examples, the electrical cabling can be Recommended Standard 232 (RS-232) cabling.

The portable pieces of equipment can include or otherwise be coupled to a network gateway hardware device that enables communication between the respective portable pieces of equipment and the production facility control system. The network gateway hardware device for each portable piece of equipment can be coupled to a communication interface of a respective skid. In addition, at least some of the skids can be logically configured to be coupled to various pieces of portable equipment. In this way, the pieces of portable equipment can be physical connected to a particular skid based on the configuration of a particular production line and the skids can be configured to operate in different configurations based on the different pieces of equipment coupled to the skid.

Additionally, the portable pieces of equipment can be coupled to at least one information communication and/or storage device, such as a dongle. The information communication and/or storage device can store information that is provided to the respective piece of equipment to which it is coupled that enables control of the respective piece of equipment via the production facility control system. The information communication and/or storage device can store information that includes one or more identifiers of a respective piece of equipment, one or more functions of the respective piece of equipment, one or more control signals corresponding to the respective piece of equipment, one or more status flags related to the respective piece of equipment, or combinations thereof. In some examples, the data stored by the information communication and/or storage device can be based at least partly on the functions, or a type, of the respective piece of equipment. In situations where a portable piece of equipment is placed in a different location along a production line and/or has a different function, the information communication and/or storage device of the portable piece of equipment can be switched to an additional information communication and/or storage device that indicates a different function and a different identifier for the portable piece of equipment.

Further, the controllers and control systems described herein can include an additional logical layer that can be used on top of conventional control software and systems. In particular implementations, the control systems described herein can include an additional abstraction layer that enables the assignment, also referred to as “binding,” of the portable pieces of equipment to various identifiers, tags, operating conditions, and flags that correspond to a specified set of functions for a specific piece of equipment at a particular location along the production line. In this way, a piece of equipment is not logically represented in the control system until the location and function of the piece of equipment is known. Thus, portable pieces of equipment can be coupled with skids in a variety of combinations without having to change the underlying control software that is being utilized to control the components of the skids and also control the portable pieces of equipment.

In illustrative examples, a production line for manufacturing a purified protein of interest (e.g., but not limited to, a purified protein drug substance) can include a first skid that includes the bioreactor (e.g., a single-use bioreactor system), a second skid that includes the TFF perfusion system, and a third skid that includes a continuous first chromatography system. The skids can be configured to couple to multiple portable pieces of portable equipment. For example, the skids can include interfaces and physical hardware to couple to portable mix tanks, filter banks, storage containers, surge vessels, holding vessels, diverter valve systems (for switching automatically switchable alternate dual flow path or multi-flow path unit operations), and/or other flow control devices. Mix tanks (or interchangeably, “mixing vessels”) or storage containers can serve as feed tanks or collection vessels, which can function as surge vessels in a continuous or semi-continuous format manufacturing process, or function as holding vessels in a batch mode format manufacturing process.

After coupling a piece of portable equipment to a skid, the piece of portable equipment can be registered with the production facility control system. The piece of portable equipment, for example, the bioreactor and/or the perfusion system and/or the low-shear (e.g., centrifugal) pump system can have a unique address that the piece of portable equipment can communicate to the production facility control system. The unique address can indicate a type of the piece of portable equipment and a unit identifier to the production facility control system. A dongle coupled to the piece of portable equipment can store an additional identifier that corresponds to a location of the skid to which the portable piece of equipment is coupled and one or more functional roles of the portable piece of equipment. For example, a mix tank can be identified as a feed tank, or as a collection tank, e.g., as the permeate receptacle, based on the location of the portable piece of equipment and the logical association of the drop to which the portable piece of equipment is coupled. In another example, a filter bank can be identified as a viral filtration device in a first configuration of a production line and then identified as a diafiltration device in a second configuration of a production line. In these situations, a first dongle can be coupled to the filter bank in the first configuration of the production line and a second dongle can be coupled to the filter bank in the second configuration of the production line. Additionally, the type of filter used in the filter bank can be changed when the filter bank is used in different locations of a production line.

In response to obtaining the information from the portable piece of equipment after being coupled to the skid, the production facility control system can determine the location and functions of the portable piece of equipment and assign the corresponding control templates to the portable piece of equipment. For example, in situations where a mix tank is functioning as a collection tank, the production facility control system can assign a first set of tags, flags, identifiers, and setpoints to the mix tank and in situations where a mix tank is functioning as a feed tank, the production facility control system can assign a second set of tags, flags, identifiers, and setpoints to the mix tank. The production facility control system can then assign a particular set of control modules to the portable piece of equipment based on the information obtained from the portable piece of equipment after being coupled to the skid.

In various embodiments, pieces of equipment that are not considered portable, such as large collection tanks can also be coupled to the skid. In these scenarios, the non-portable pieces of equipment may not include the hardware and/or communication and storage devices that enable dynamic configuration of the non-portable piece of equipment with respect to the production facility control system. If the non-portable piece of equipment is not configured for a dynamic configuration, an operator of the production facility control system can manually establish the template and/or control module used to control the operation of the non-portable piece of equipment.

In addition to the control of the pieces of equipment included in a production line, the production facility control system can also track the decay rate of a batch during production of a purified protein of interest (e.g., but not limited to, a purified protein drug substance). The “decay rate” is a period of time in which materials used for the production of sub-lots can be identified and tracked. For example, the materials used (e.g., buffers, cell culture medium, etc.) in a resulting chromatography step eluate pool collection, of which there may be many, can be identified and tracked in a dynamic fashion by way of the “decay rate.” In a continuous batch production process, the production facility control system can estimate the decay rate for the purified protein production process. In various implementations, the production facility control system can assign batch identifiers to certain portions of the production of the batch and initiate a decay monitor until the current batch identifier is changed to a new batch identifier and a new decay monitor is implemented for the new batch identifier.

In an illustrative example, the production facility control system can determine that a filter bank (serving in this example as permeate receptacle) is coupled between an automated TFF perfusion bioreactor of the invention and a first chromatography system based on information obtained from a dongle coupled to the filter bank. In these situations, the filter bank can operate as a depth filter. The production facility control system can identify one or more control modules, flags, and/or status identifiers for a depth filter and execute the one or more control modules while the filter bank is being used in a production line. The production facility control system can monitor pressure within the filter assemblies of the filter bank based on pressure values obtained from pressure sensors included in the filter assemblies. The production facility control system can determine that the pressure within a first filter assembly through which material is flowing has reached at least a threshold level. The threshold level of pressure can indicate that a filter included in the first assembly needs to be replaced due to a decrease in the amount of material that can be processed by the filter. The production facility control system can then send a signal to control a diverter valve coupled to the filter bank to cause the material to flow through a second filter assembly of the filter bank. The filter included in first filter assembly can then be replaced.

By way of further illustration, the following embodiments of the present invention are enumerated:

Embodiment 1: An automated tangential flow filtration (TFF) perfusion bioreactor, comprising:
(a) a bioreactor configured for culturing eukaryotic cells, the bioreactor having an inlet for receiving volumes of fresh cell culture medium;
(b) a perfusion system comprising a recirculation loop fluidly linked with the bioreactor through a first port of the recirculation loop and a second port of the recirculation loop, the recirculation loop comprising, between the first port and the second port, a hollow fiber filter comprising a lumen having a porous surface capable of preventing the passage of whole cells but allowing volumes of conditioned cell culture medium to pass and to exit the perfusion system into a permeate receptacle, wherein volumes of conditioned medium containing cells from the bioreactor can enter through the first port into the recirculation loop, and flow tangentially in relation to the porous surface, and a retentate can flow out from the recirculation loop through the second port back into the bioreactor;
(c) a low-shear pump system comprising at least one pump head that directs the flow of the conditioned medium containing cells through the recirculation loop in a first direction across the porous surface of the hollow fiber filter and, interchangeably, in a second direction, opposite the first direction; and
(d) a controller capable of automatically switching the low-shear pump system to direct the flow of the conditioned medium containing cells through the recirculation loop in the first direction, and after a predetermined period, automatically switching the low-shear pump system to direct the flow of the conditioned medium containing cells to the second direction, and vice versa, wherein the first and the second directions are periodically exchanged and the conditioned medium containing cells synchronously enters the recirculation loop from the bioreactor through the second port instead of the first port and the retentate returns to the bioreactor through the first port instead of the second port, and vice versa;
wherein the TFF perfusion bioreactor is configured to operate over a production cultivation period of 13-90 days at a viable cell density of about 60-300×10⁶/mL, and wherein sieving is improved when compared to an equivalently-scaled non-switching system.

Embodiment 2: The automated tangential flow filtration perfusion bioreactor of Embodiment 1, wherein the low shear pump system comprises one or more centrifugal pumps.

Embodiment 3: The automated tangential flow filtration perfusion bioreactor of any of Embodiments 1-2, wherein the controller is configured to automatically switch the direction of flow through the low-shear pump system after the predetermined period of about 20 minutes, or about 1 hour, or about 6 hours, or about 12, hours, or up to about 24 hours.

Embodiment 4: The automated tangential flow filtration perfusion bioreactor of any of Embodiments 1-3, wherein the perfusion bioreactor is configured for culturing mammalian cells, insect cells, algal cells, or yeast cells.

Embodiment 5: The automated tangential flow filtration perfusion bioreactor of any of Embodiments 1-4, wherein the perfusion bioreactor is configured for culturing mammalian cells.

Embodiment 6: The automated tangential flow filtration perfusion bioreactor of any of Embodiments 1-5, wherein the TFF perfusion bioreactor is configured to operate over a production cultivation period of 20-60 days.

Embodiment 7: The automated tangential flow filtration perfusion bioreactor of any of Embodiments 1-6, wherein the TFF perfusion bioreactor is configured to operate at a viable cell density of about 100-150×10⁶/mL.

Embodiment 8: The automated tangential flow filtration perfusion bioreactor of any of Embodiments 1-7, wherein the bioreactor is configured for single-use.

Embodiments 9: The automated tangential flow filtration perfusion bioreactor of any of Embodiments 1-8, wherein the recirculation loop is configured for single-use.

The following working examples are illustrative and not to be construed in any way as limiting the scope of the invention.

### EXAMPLES

### Example 1. Comparison of Low-Shear Bidirectional TFF with Unidirectional TFF Systems

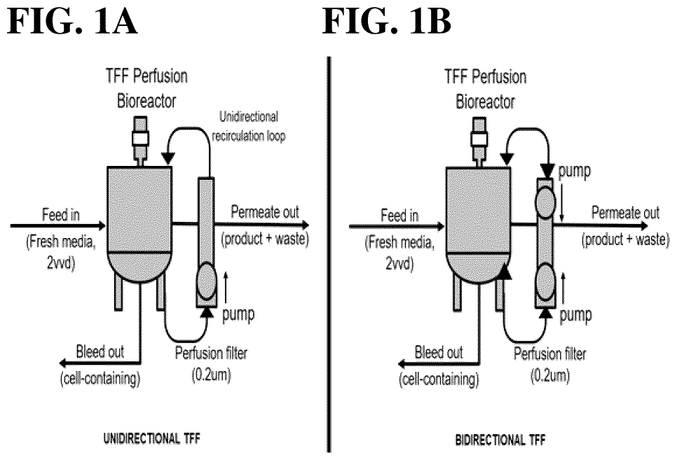

An experiment was performed to test an embodiment of the inventive automated tangential flow filtration (TFF) perfusion bioreactor, including a centrifugal pump-based bidirectional TFF system, as described hereinabove. This embodiment used two Levitronix® TFF systems controlled remotely through Ignition (an integrated, flexible automation platform for supervisory control and data acquisition (SCADA); Inductive Automation) to periodically switch the direction of flow in the TFF recirculation loop on a predetermined cycle time. This system is depicted schematically in FIG. 1B. In this experiment, three different predetermined cycle times were tested: 10 minutes, 20 minutes, and 60 minutes. The bidirectional flow conditions were compared with a unidirectional flow control, using a single low-shear centrifugal pump. Additionally, a condition was run using a high-shear peristaltic pump (Masterflex® L/S®; Cole-Parmer) run unidirectionally in comparison to the low-shear centrifugal system run unidirectionally.

### Materials and Methods.

Vial Thaw and Seed Train Expansion. The purpose of vial thaw and cell culture expansion in shake flasks is to generate sufficient inoculum volume for seeding the bioreactor N−1 (typically a 2-L to 5-L working volume). One 1-mL vial of a cell bank of CHO cells that produce an antibody was thawed in hand and added to a disposable, non-baffled, vented shake flask containing the seed train medium for a total final volume of approximately 30 mL at 1×10⁶ cells/mL. Then, shake flask cultures were incubated in humidified, temperature and CO₂-controlled shaking incubators; CO₂ was controlled to 5% (v/v). The initial passage was incubated for 2 days, and all subsequent passages were incubated for 3 days (see, Table 1.1).

Cultures were expanded in disposable, non-baffled, vented shake flasks of increasing size until sufficient inoculum was available for seeding the N−1 bioreactor. At each passage, confluent cultures were counted on a Vi-Cell XR to ensure high viability (>97%) and sufficient growth to seed the next stage.

N−1 Bioreactor Operation. The N−1 was operated in batch mode at a working volume of 5 L, with the aim of providing enough inoculum volume to seed the production reactors. The N−1 was a 3-day culture with the setpoints as described in Table 1.1, below.

TABLE 1.1
N-1 Culture Parameters & Setpoints.

| Parameter | Value | Units |
|---|---|---|
| Batch Medium | Ex-Cell Advanced CHO | N/A |
| | Fed Batch Medium | |
| pH | 7.0 +/− 0.02 | N/A |
| Temperature | 36.8 | ° C. |
| Dissolved Oxygen | 60 | % air |
| (DO) | | saturation |
| Agitation | 316 | RPM |
| Target Inoculation | 5 × 10⁵ | viable |
| Cell Density | | cells/mL |
| Batch Working | 5.0 | L |
| Volume | | |
| Air Overlay | 180 | sccm |

Production (N Stage) Bioreactor Operation. The production stage bioreactors were used to test the intensified protein production process with a high-performance cell line and an optimized intensified protein manufacturing process typical of an industry production platform. Productions were run in 1.6-L-working volume glass and stainless-steel vessels using a typical TFF perfusion hollow fiber filter and low-shear centrifugal TFF pump and controller. Operating parameters and setpoints are shown in Table 1.2, below.

TABLE 1.2
Production culture parameters and setpoints.

| Parameter | Value | |
|---|---|---|
| Cell Line/Molecule | Horizon CHO-K1 clone | |
| | producing an antibody | |
| Batch Medium | Ex-Cell Advanced CHO | |
| | Fed Batch Medium | |
| Bioreactor Type | Applikon glass w/stainless | |
| | parts (autoclavable) | |
| Digital Control | Applikon ADI 1010 | |
| Unit | | |
| pH | 6.90 ± 0.02 | |
| Base type | 1M Sodium Carbonate | |
| Acid type | CO₂ gas | |
| Perfusion Media | Chemically-Defined | |
| | Perfusion Media | |
| Perfusion Mode | Continuous Harvest | |
| Perfusion | TFF | |
| Recirculation | | |
| Method | | |
| Perfusion Filter | 0.09 m² Microza ® (Asahi Kasei) | |
| | with recycle | |
| Flux per day at | 112.5 | L/L/m²/d |
| 2VVD (Permeate + | | |
| Recycle) | | |
| Recirculation | Leviflow ® LFSC-D Series | |
| flow sensor | | |
| Pressure sensors | Pendotech PREPSN038 3/8″ | |
| | hose barb sensors | |
| Recirculation Flow | 1.6 | L/min |
| Rate (Crossflow) | | |
| Shear Inside Filter | 990 | s⁻¹ |
| Sintered Sparger | 15 | μm |
| Size | | |
| Sparger Switch | When required - between | |
| Day | days 6-8 | |
| pH | 6.90 ± 0.02 | |
| Temperature | 36.8° | C. |
| Dissolved oxygen | 60% air saturation | |
| (DO) | | |
| Agitation | 435 | RPM |
| Target Inoculation | 6 × 10⁵ viable cells/mL | |
| Cell Density | | |
| Target VCD | 120 × 10⁶ viable cells/mL | |
| Batch Working | 1.25 L + 0.25 L to prime and | |
| Volume | soak perfusion filter | |
| Nominal Working | 1.6 | L |
| Volume | | |
| Air Overlay | 60 | sccm |
| Background Air | 60-175 | sccm |
| Sparge | | |

| | |
|---|---|
| Background Air | Same as sparger switch |
| Sparge Start Day | day (see above) |

Bioreactor pH was controlled on one side of the deadband with sparged CO₂ through the same pipe as O₂ delivery, and on the other side of the deadband by addition of 1 M sodium carbonate. In-process 1-point calibration of pH was carried out if the offline pH measurement was more than 0.03 pH units from the probe value. Dissolved oxygen was maintained by pure O₂ delivered to the drilled hole pipe, then sintered sparger when increased gas demand required it. A background air sparge delivered air through the drilled pipe throughout the run (starting on day 0) in order to strip CO₂.

Starting mid-run, 50% glucose was added to the bioreactor on an hourly timed drip using a peristaltic pump. The glucose feed was started on the day glucose dipped below 2.5 g/L when measured offline, and the pump rate was adjusted to maintain sufficient levels throughout the run.

A continuous cell bleed was implemented mid-run to maintain cells at about 120×10⁶ cells/mL. Cell bleed was calculated independent of perfusion rate (such that a 10% bleed led to media addition equivalent to 2.1 vvd).

Gamma-irradiated EX-CELL® Antifoam (SAFC) was added on an hourly drip using a timed pump. Antifoam addition started between days 2 and 5 starting at 0.2 mL/hour and was increased as needed to maintain a minimal head of foam on the culture.

Daily sampling was performed using a Nova BioProfile® FLEX2 for pH, CO₂, analytes such as lactate, glucose, and ammonium, and osmolality. Viable cell density (VCD), viability, and cell diameter snapshots were taken daily on a Vi-Cell XR. Packed cell volume (PCV) was measured regularly using specialized cuvettes. Finally, daily retains were saved from both the bioreactor and permeate; titer from these samples was used to calculate productivity (qP) and sieving throughout the run.

Perfusion Operation. Perfusion was controlled for the centrifugal TFF conditions (both bidirectional and unidirectional) using a Levitronix® pump console LCO-I100 and associated Levitronix® PuraLev®i100SU pump drive system(s). Perfusion for the peristaltic condition was controlled using a Cole-Parmer Masterflex® L/S® pump with an appropriately-sized pump-head for the recirculation tubing.

The perfusion hollow fiber filter used for all conditions was a Microza® 0.2-μm pore size filter with 0.09 m² surface area (Asahi Kasei, Part No. UMP-1047R).

For all conditions, a recycle pump was used to scale the total permeate flux through the TFF filter to the equivalent flux of a 450-L working volume bioreactor with an appropriately-scaled filter. This was done by adding a two-pronged branching connector (i.e., a “Y”) to the permeate line; one arm of the “Y” was fed back into the pump at a continuous flow rate, while the other arm was fed into the permeate bag at the correct VVD. Table 1.3 shows the flux rates used to generate recycle rates for a 1.6-L working volume.

TABLE 1.3
Perfusion ramp schedule.

| | Perfusion Rate, | Total Flux | Ideal | Ideal |
|---|---|---|---|---|
| | Vessel Volumes | per Day | Permeate Rate | Recycle Rate |
| Day | per Day (VVD) | (L/m2/d) | (mL/min) | (mL/min) |
| 0-2 | 0.0 | 0.00 | 0.00 | 0.00 |
| 2-3 | 0.5 | 28.13 | 0.56 | 1.23 |
| 3-4 | 1.0 | 56.25 | 1.12 | 2.46 |
| 4-5 | 1.5 | 84.38 | 1.68 | 3.69 |
| 5+ | 2.0 | 112.5 | 2.24 | 4.93 |

### Results and Discussion

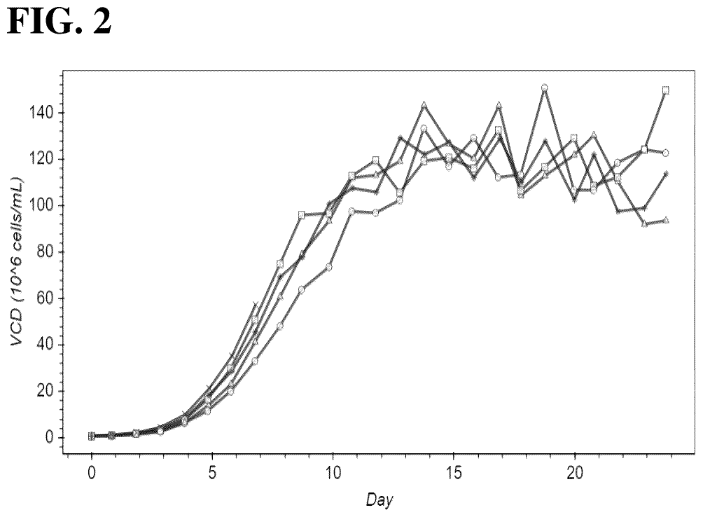

Overall, the cells in the bioreactors grew well across all experimental conditions, with VCDs reaching target density (120×10⁶ cells/mL) between days 11 and 13 (except for the peristaltic pump-driven condition, which exhibited catastrophic failure before the target density was reached; see, further discussion below). Viable cell densities remained at the target density consistently, with a small cell bleed employed, until the end of the experimental run (FIG. 2). Packed cell volume (FIG. 3) was also measured to be consistent across experimental conditions.

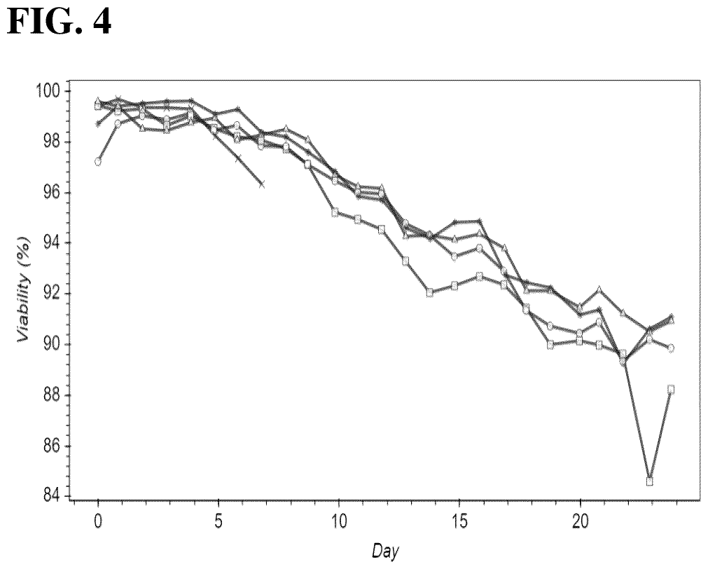

As noted above, the peristaltic pump-driven condition failed early. Aside from early viability drops, the silicone tubing in the peristaltic pump head tore open on days 6-7, requiring the bioreactors under this condition to be shut down. Viability (FIG. 4) was significantly lower in the peristaltic pump-driven condition compared with the other conditions by day 5, and continued to decline until the reactors had to be shut down. There was no significant difference in viability between the unidirectional flow and any of the bidirectional flow conditions, when flow through the recirculation loop was driven by a low-shear (centrifugal) pump.

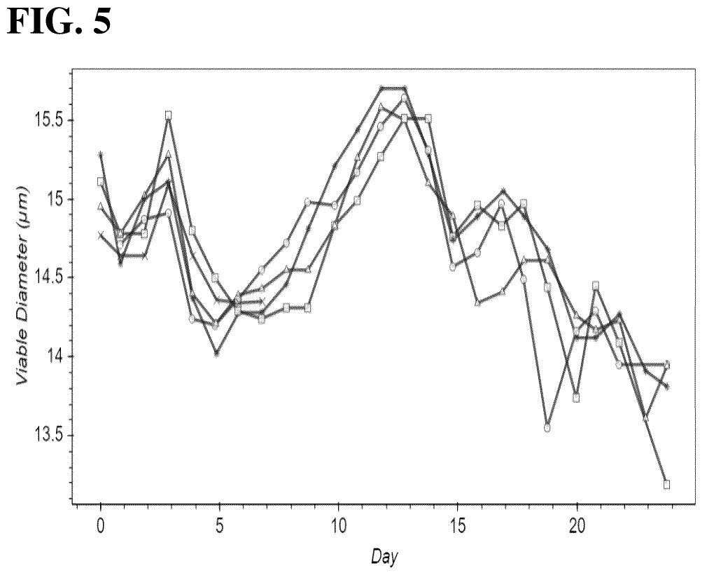

There was no difference in viable cell diameter across experimental conditions; however, all conditions followed a somewhat unusual trend over the course of the run (FIG. 5). At the start of TFF perfusion the cells declined in size, then increased until about day 13; after this, cells continued to decline in average diameter until the end of the run. This decline coincided with a decline in productivity. The histograms showing cell size distribution (not shown) changed shape over time, as well, gradually becoming wider and bimodal.

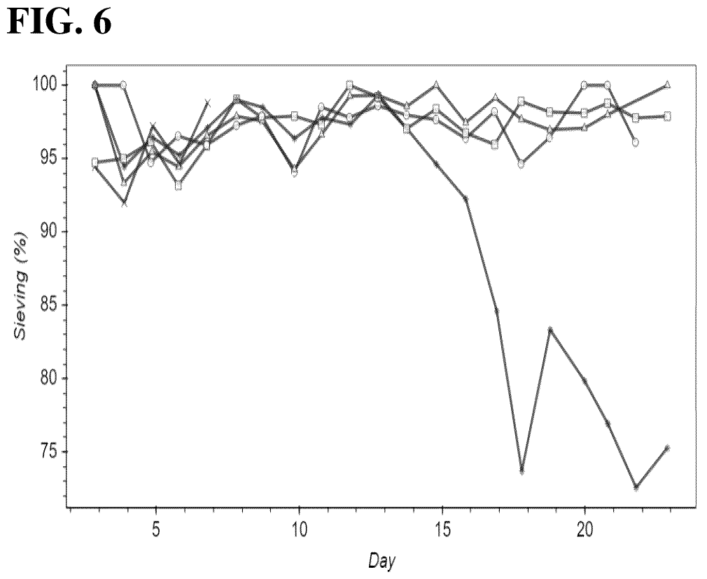

The clearest difference in performance between experimental conditions was seen in sieving (FIG. 6). The unidirectional flow condition exhibited a sieving decline starting around day 14; sieving eventually dropped to about 70% by the end of the run after 23 days. In contrast, under all bidirectional flow conditions sieving was maintained between 90% and 100% until the end of the run.

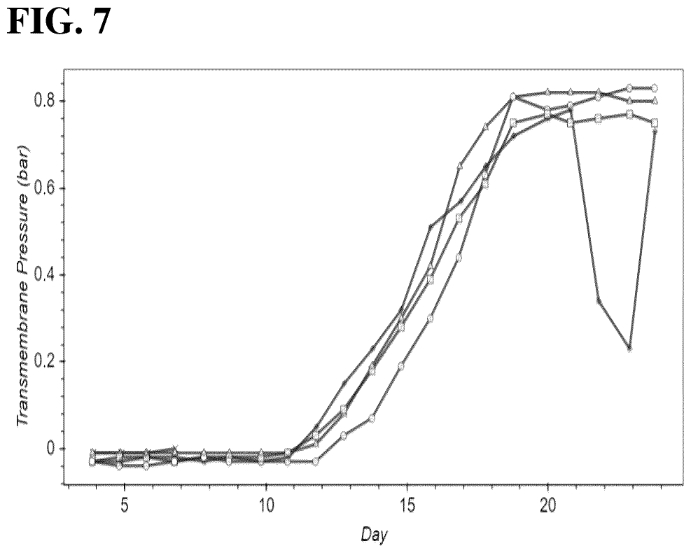

Interestingly, sieving and TMP (FIG. 7) did not correspond with each other. All bioreactor conditions exhibited a steady increase in TMP starting around day 12 and cavitation in their permeate lines around the same time; there was no difference between the inventive bidirectional and unidirectional flow conditions. Bioreactors were eventually shut down when they were no longer able to pass permeate at 2VVD with a consistent permeate pump rate; no reactors made it past day 24. A caveat on the unidirectional flow condition was that the recycle pump was accidentally turned off on days 22-23, so the flux was much lower than expected and there was a temporary drop in TMP. When the recirculation loop recycle was restarted for this bioreactor, TMP increased to its previous level and cavitation was once again seen.

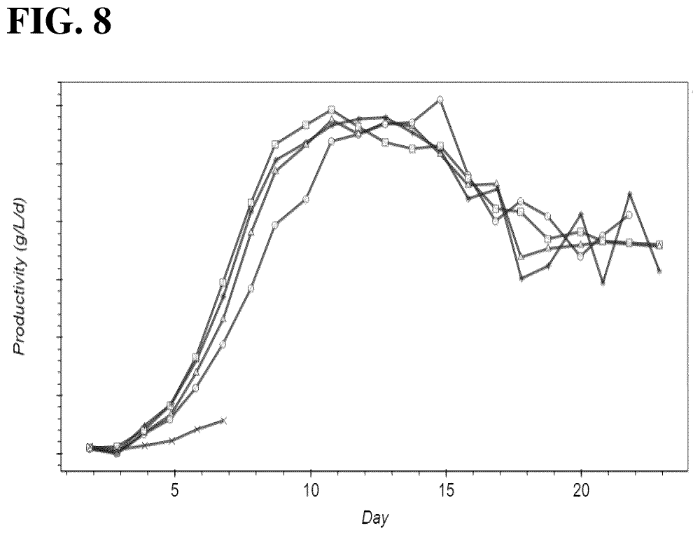

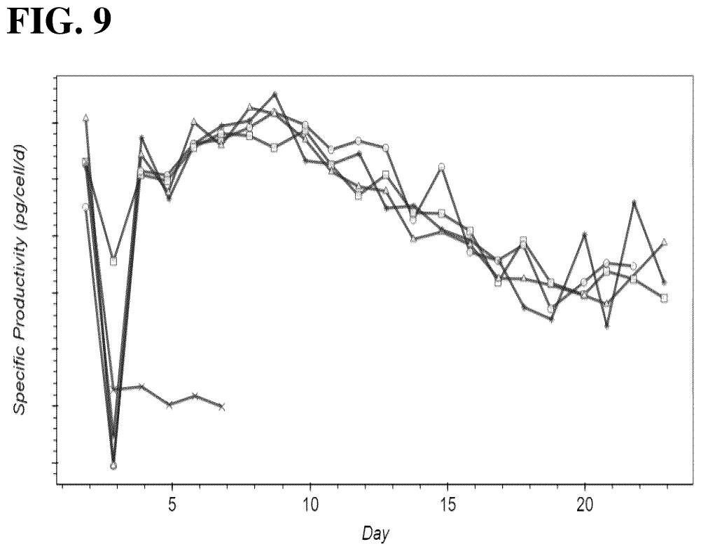

Even in the first 7 days of culture, while all conditions were growing similarly, the experimental conditions employing a low-shear (centrifugal) pump to drive flow through the recirculation loop greatly outperformed the peristaltic pump-driven conditions in titer of protein production (FIG. 8). This difference between centrifugal pump driven conditions versus peristaltic pump-driven conditions was even more obvious in the specific productivity (FIG. 9); the peristaltic pump-driven conditions produced only ⅕ the product that the centrifugal conditions did on a per-cell basis. No difference in productivity, or qP, was seen between the unidirectional flow and any bidirectional flow experimental conditions.

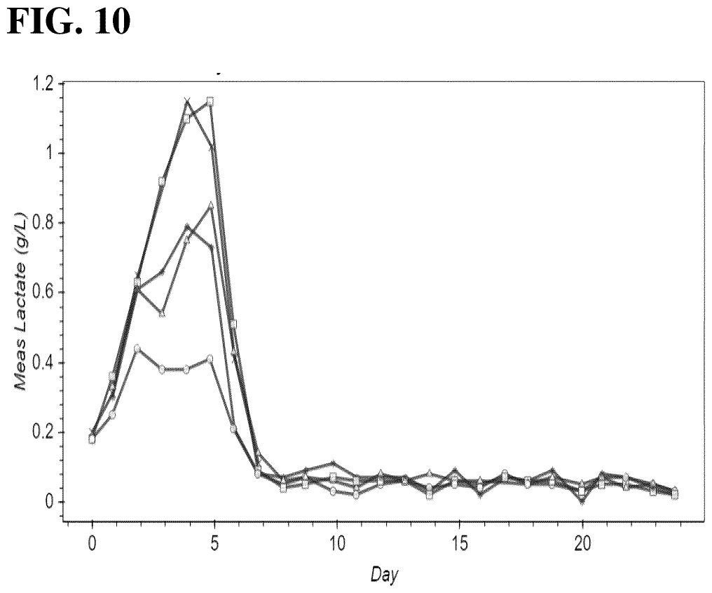

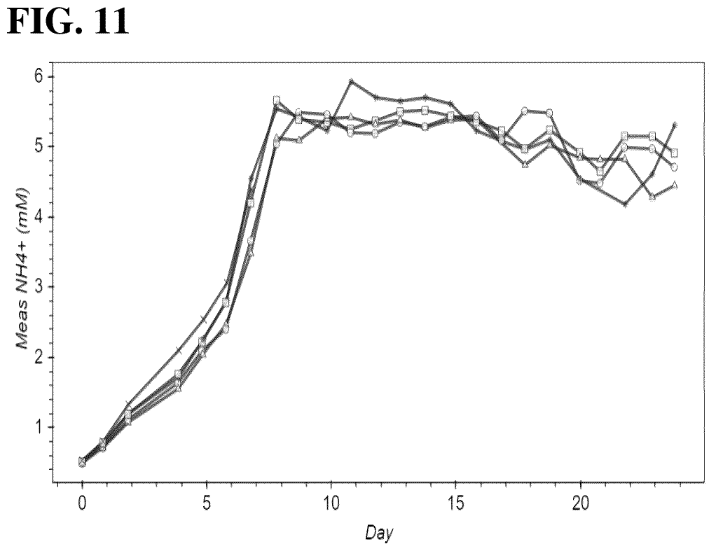

Lactate concentration in the conditioned culture medium was measured offline daily, according to the instrument manufacturer's protocol using a BioProfile® FLEX2 Automated Cell Culture Analyzer (Nova Biomedical) with chemistry sensors and a chemistry analysis component; lactate concentration followed a typical trend, spiking in the growth phase of the culture but coming back down around day 7 and remaining very low throughout the rest of the run (FIG. 10). Similarly, ammonium concentration in the conditioned culture medium, also measured on the BioProfile® Flex 2 instrument (Nova Biomedical) according to the instrument manufacturer's protocol, increased during the growth phase but then leveled off at a steady level until the end of the run (FIG. 11]).

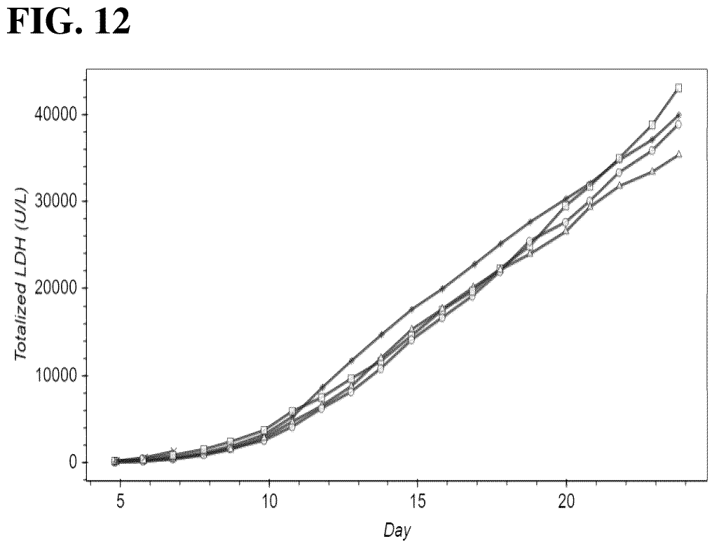

Lactate dehydrogenase (LDH) enzyme in the conditioned culture medium was also measured offline using a Cedex BioHT (Roche) chemistry analyzer according to the manufacturer's protocol, as an indicator of cell death. Interestingly, totalized LDH was consistent across the experimental run; there was no significant difference in totalized LDH between the bidirectional and unidirectional flow conditions (FIG. 12).

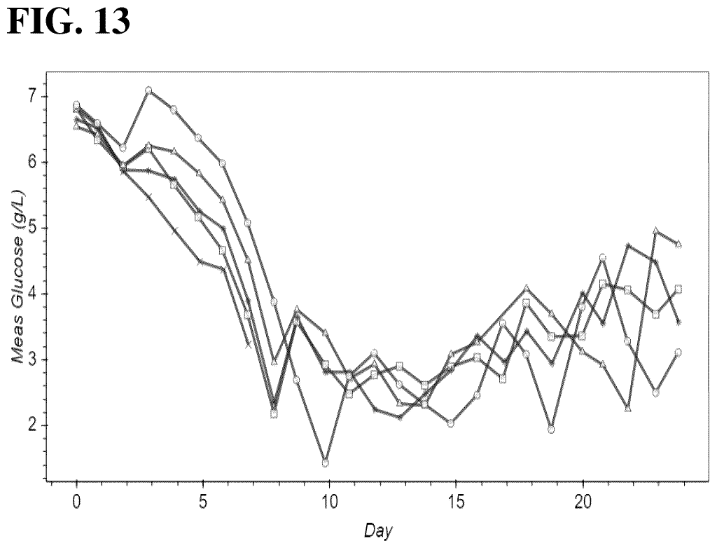

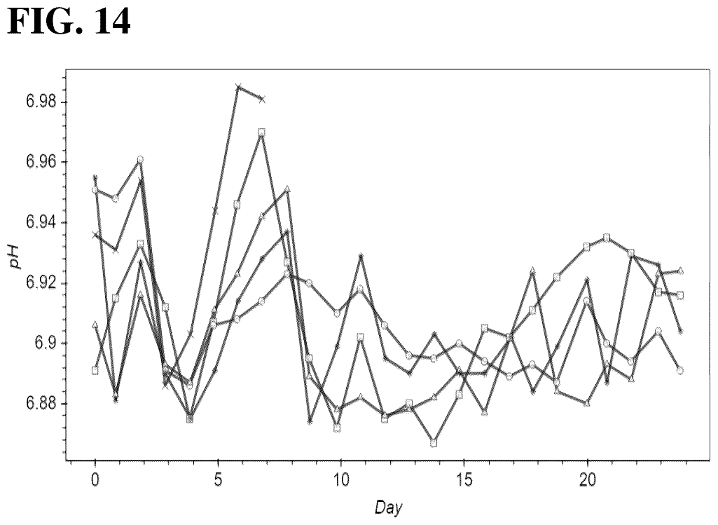

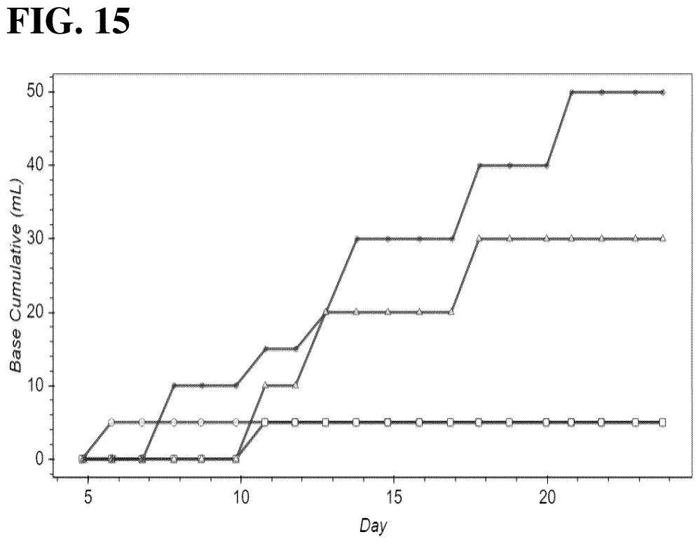

Glucose concentration in the conditioned culture medium was maintained within an acceptable range throughout the run (FIG. 13). The pH (FIG. 14) remained largely within the deadband (6.88-6.92), requiring periodic pH 1-point in-process calibrations (a common calibration procedure to align online and offline pH measurements) early in the experimental run but becoming more consistent towards the end of the run. Bioreactors required relatively little base addition to maintain the pH setpoint (FIG. 15).

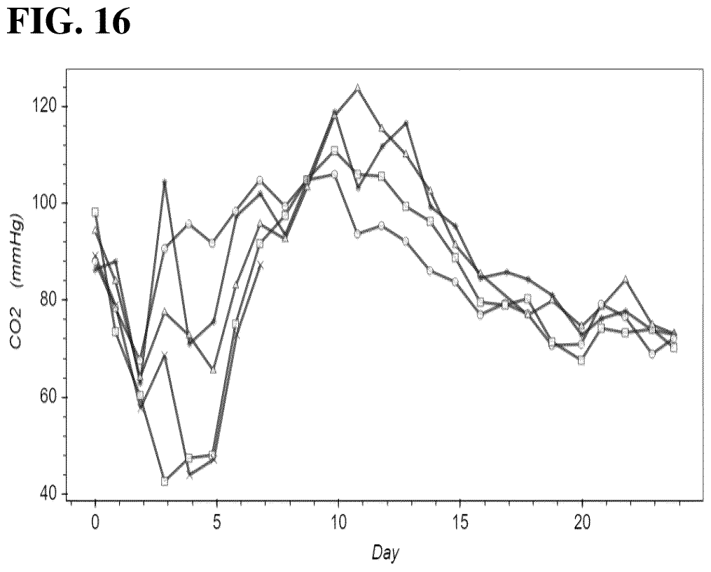

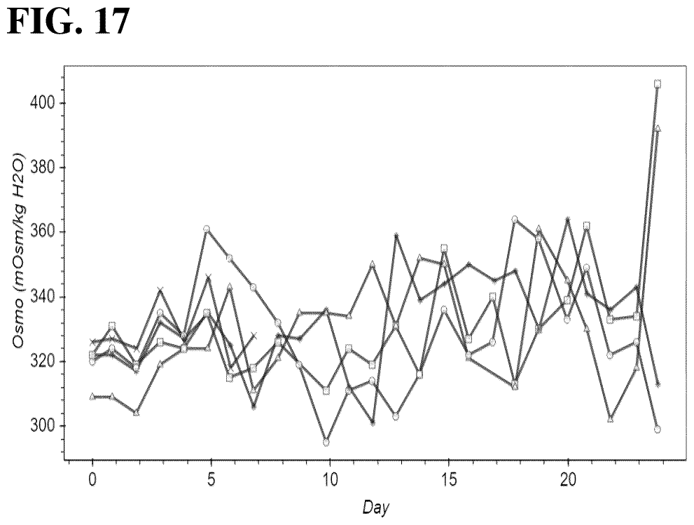

The concentration of dissolved CO₂ remained within an acceptable range, reaching no higher than about 120 mmHg (FIG. 16). Osmolality remained mostly between about 300 and 360 mOsm/kg of solvent water for all conditions tested (FIG. 17).

The experiment in this Example 1 successfully showed that the inventive bidirectional flow TFF system using centrifugal pump systems was advantageous over the unidirectional flow TFF perfusion systems commonly employed in the biologics industry. Here, the peristaltic pump-driven flow conditions had early (at about day 5) drops in viability and ⅕ the per-cell productivity that the other conditions had. Moreover, in the experimental conditions employing a peristaltic pump to drive flow through the recirculation loop, the silicone recirculation tubing consistently ruptured by day 7; this was a catastrophic failure that required the bioreactors to be shut down. The use of a low-shear (e.g., centrifugal) pump system entirely eliminates the risk of catastrophic failure that was seen with the use of peristaltic pumps.

More importantly, the bidirectional flow system had significantly better sieving until the end of the run (remaining above 90% the entire run) compared to the unidirectional flow using a centrifugal pump system, which dropped as low as 70%. Finally, bidirectional cycle time did not affect filter performance within the 10-minute to 60-minute timeframe examined in this Example 1 for alternating the flow direction in the recirculation loop.

### Example 2. Another Comparison of Low-Shear Bidirectional TFF with Unidirectional TFF Systems

Another experiment was performed to test an embodiment of the invention, including a centrifugal pump-based bidirectional TFF system, as described hereinabove. This embodiment used two Levitronix® TFF systems controlled remotely through Ignition (an integrated, flexible automation platform for supervisory control and data acquisition (SCADA); Inductive Automation) to periodically switch the direction of flow in the TFF recirculation loop on a predetermined cycle time. This system is depicted schematically in FIG. 1B.

A cycle time of 20 minutes was used for alternating the direction of flow in the recirculation loop. The bidirectional flow condition was compared with a unidirectional flow control, using a single low-shear centrifugal pump. This experiment used a hollow fiber filter with different characteristics from the perfusion filter used in Example 1 (e.g., sieving in particular) with high-density cell cultures in their process, with the purpose of determining whether the inventive bidirectional flow TFF system improved sieving across different filter types.

For this experiment, vial thaw and seed train expansion, N−1 bioreactor operation, production (N stage) bioreactor operation and sampling were conducted as described in Example 1, above. Parameters and setpoints for N−1 culture were the same as those described in Table 1.1, hereinabove; operating parameters and setpoints for the production culture are shown in Table 2.1, below.

Perfusion operation was controlled as described in Example 1 hereinabove, except that the perfusion filter used for both conditions in this Example 2 was the Spectrum® 0.2-μm pore size MiniKros® hollow-fiber filter with 0.047 m² surface area (Repligen, Part No. S02-P20U-10-N). For all conditions, a recycle pump was used to scale the total permeate flux through the filter to the equivalent flux of a 450-L working volume reactor with an appropriately-scaled filter, as described in Example 1, hereinabove. Table 2.2, below, shows the flux rates used to generate recycle rates for a 1.6-L working volume.

TABLE 2.1
Production culture parameters and setpoints.

| Parameter | Value | |
|---|---|---|
| Cell Line/Molecule | Horizon CHO-K1 clone | |
| | producing an antibody | |
| Batch Medium | Ex-Cell Advanced CHO | |
| | Fed Batch Medium | |
| Bioreactor Type | Applikon glass w/stainless | |
| | parts (autoclavable) | |
| Digital Control Unit | Applikon ADI 1010 | |
| pH | 6.90 ± 0.02 | |
| Base type | 1M Sodium Carbonate | |
| Acid type | CO₂ gas | |
| Perfusion Media | Chemically-Defined | |
| | Perfusion Media | |
| Perfusion Mode | Continuous Harvest | |
| Perfusion | TFF | |
| Recirculation Method | | |
| Perfusion Filter | 0.047 m² Repligen Spectrum ® Hollow | |
| | Fiber 0.2 um filter w/recycle | |
| Flux per day at | 118.42 | L/m²/d |
| 2VVD (Permeate + | | |
| Recycle) | | |
| Recirculation flow | Leviflow ® LFSC-D Series | |
| sensor | | |
| Pressure sensors | Pendotech PREPSN038 3/8″ | |
| | hose barb sensors | |
| Recirculation Flow | 1.6 | L/min |
| Rate (Crossflow) | | |
| Shear Inside Filter | 1358 | s⁻¹ |
| Sintered Sparger Size | 15 | μm |
| Sparger Switch | When required - between | |
| Day | days 6-8 | |
| Temperature | 36.8° | C. |
| DO | 60% air saturation | |
| Agitation | 435 | RPM |
| Target Inoculation | 6 × 10⁵ viable cells/mL | |
| Cell Density | | |
| Target VCD | 120 × 10⁶ viable cells/mL | |
| Batch Working | 1.25 L + 0.25 L to prime and | |
| Volume | soak perfusion filter | |
| Nominal Working | 1.6 | L |
| Volume | | |
| Air Overlay | 60 | sccm |
| Background Air | 60-175 | sccm |
| Sparge | | |

| | |
|---|---|
| Background Air | Same as sparger switch |
| Sparge Start Day | day (see above) |

TABLE 2.2
Perfusion ramp schedule.

| | Perfusion Rate, | Total Flux | Ideal | Ideal |
|---|---|---|---|---|
| | Vessel Volumes | per Day | Permeate Rate | Recycle Rate |
| Day | per Day (VVD) | (L/m2/d) | (mL/min) | (mL/min) |
| 0-2 | 0.0 | 0.00 | 0.00 | 0.00 |
| 2-3 | 0.5 | 29.61 | 0.56 | 0.41 |
| 3-4 | 1.0 | 59.21 | 1.12 | 0.82 |
| 4-5 | 1.5 | 88.82 | 1.68 | 1.23 |
| 5+ | 2.0 | 118.42 | 2.24 | 1.64 |

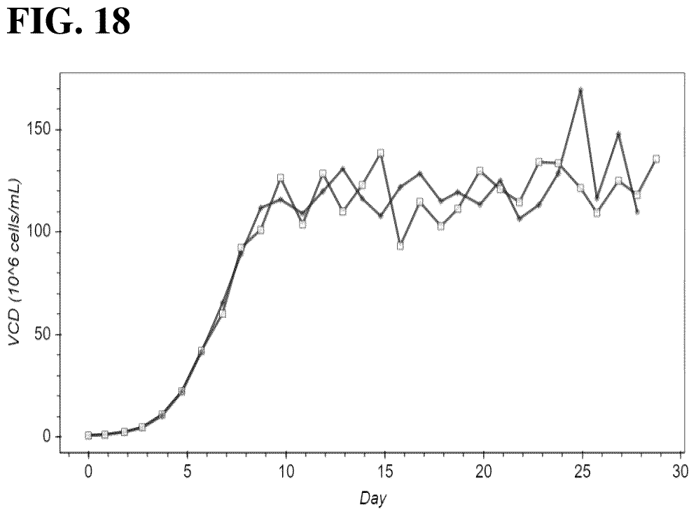

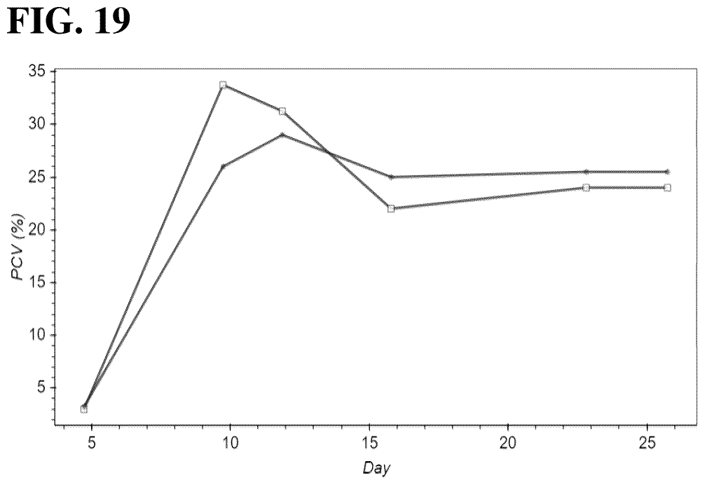

Results and Discussion. Overall, under both the unidirectional and bidirectional recirculation loop flow test conditions cells grew well, with VCDs reaching target density (120×10⁶ cells/mL) around day 10 and remaining there consistently with a small bleed until the end of the run (FIG. 18). Packed Cell Volume (FIG. 19) was also consistent for both test conditions.

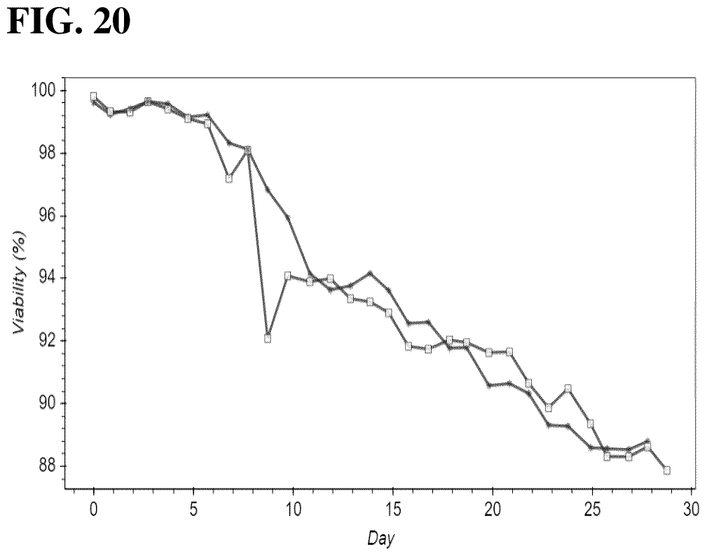

Viability remained greater than 87% throughout the run for both conditions (FIG. 20). The unidirectional flow condition had a small but sharp drop in viability on days 8-9, but for the rest of the run viability was similar under either of the conditions.

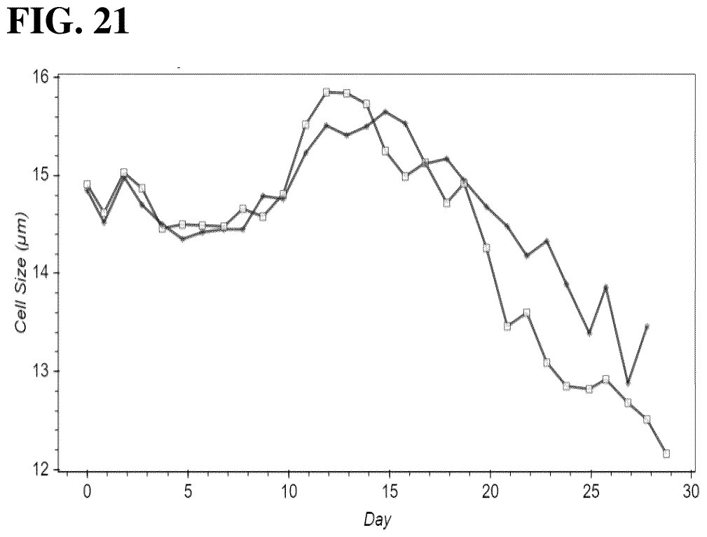

There was no difference in cell diameter across conditions; however, characteristic of this cell line, the cell size changed noticeably throughout the run (FIG. 21). At the start of perfusion, cells declined in size, then increased until about day 13; after this, they continued to decline in average diameter until the end of the run. This size decline coincided with a decline in productivity. The histograms showing cell size distribution (not shown) changed shape over time as well, gradually becoming wider and bimodal. These changes were more dramatic in the unidirectional flow condition compared with the bidirectional flow condition.

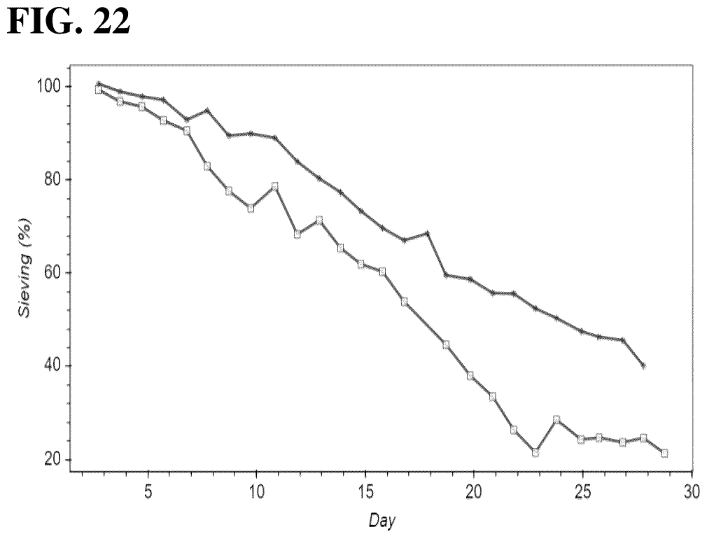

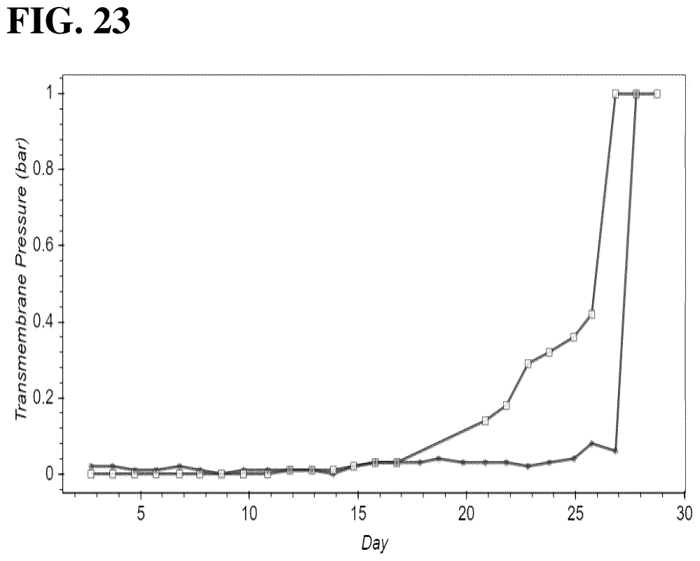

Both conditions had an obvious decline in sieving starting very early in the run and continuing downward at a steady rate. As in Example 1, the bidirectional flow condition had significantly higher sieving throughout the run compared to the unidirectional flow condition (FIG. 22). Furthermore, unlike the results in Example 1, transmembrane pressure (TMP) was also significantly lower for the bidirectional flow condition, with the unidirectional flow condition exhibiting increasing TMP around day 21 and the bidirectional flow condition exhibiting increasing TMP only after day 27 (FIG. 23).

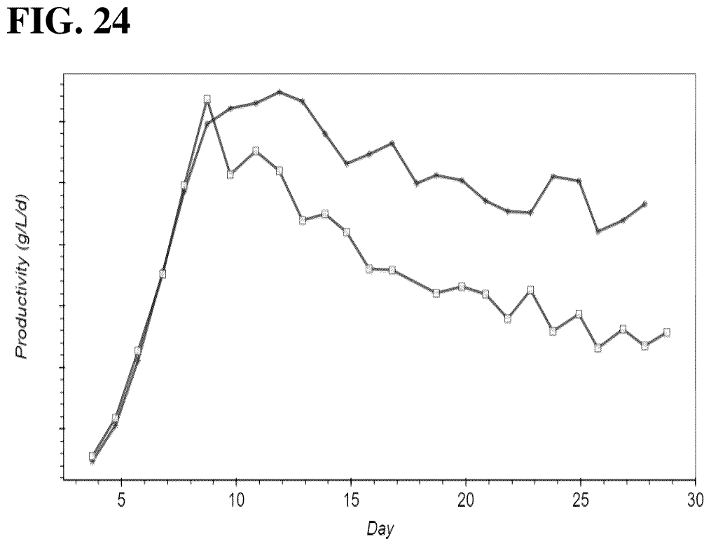

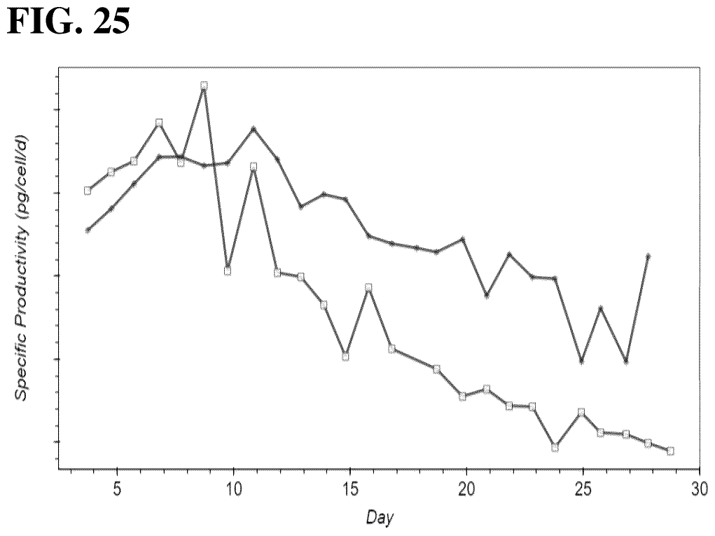

Unexpectedly, the bidirectional flow condition had somewhat higher productivity (FIG. 24) and cell-specific productivity (FIG. 25) than the unidirectional flow condition.

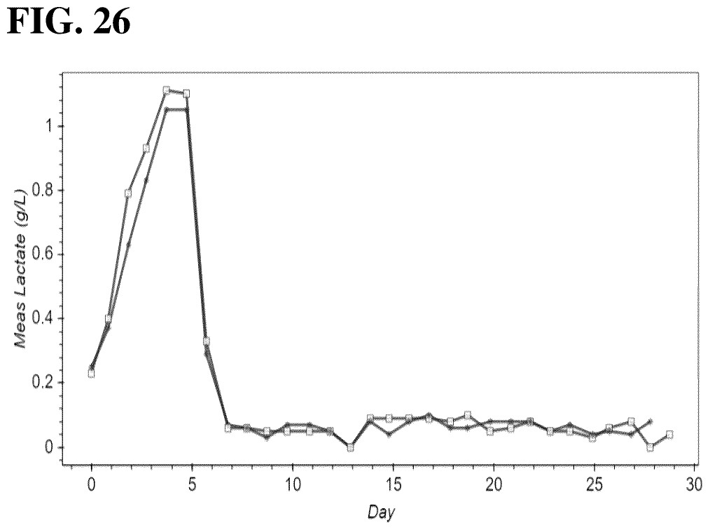

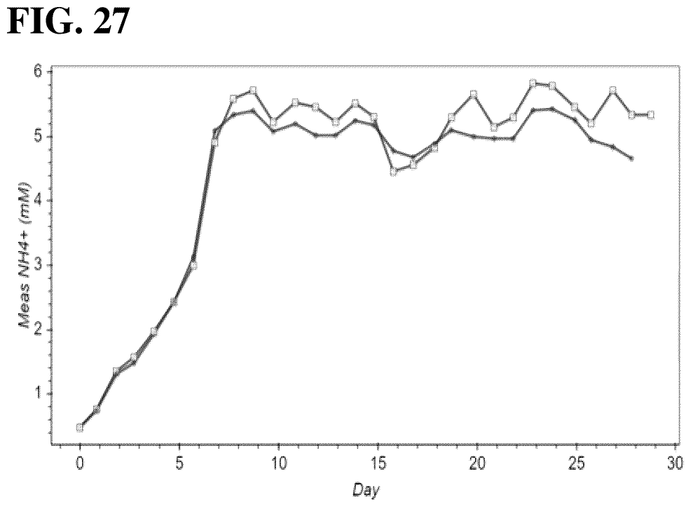

Lactate concentration in the culture medium followed a typical profile, peaking towards the beginning of the run during the growth phase and then remaining very low for the rest of the run (FIG. 26). Ammonium concentration in the culture medium generally increased during the growth phase and then leveled off for the rest of the run in the typical range see in previous experiments with this process (FIG. 27).

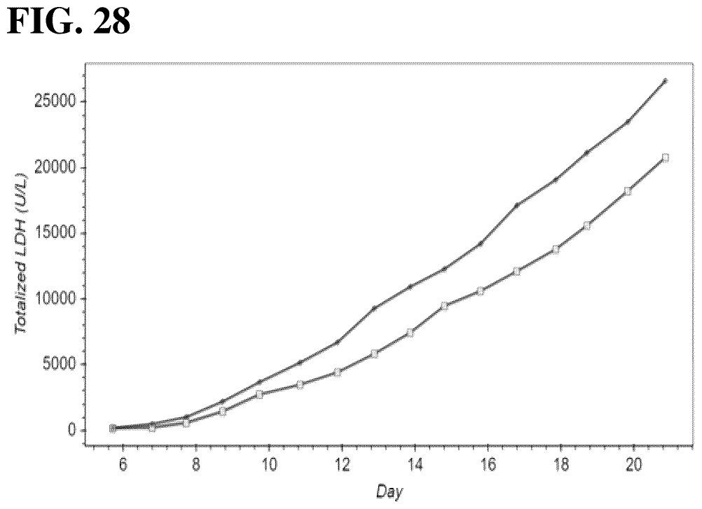

As in the experiment described in Example 1, lactate dehydrogenase (LDH) enzyme was detected in the culture medium consistently throughout the experimental run in this Example 2 (FIG. 28); totalized LDH was slightly lower for the unidirectional flow control.

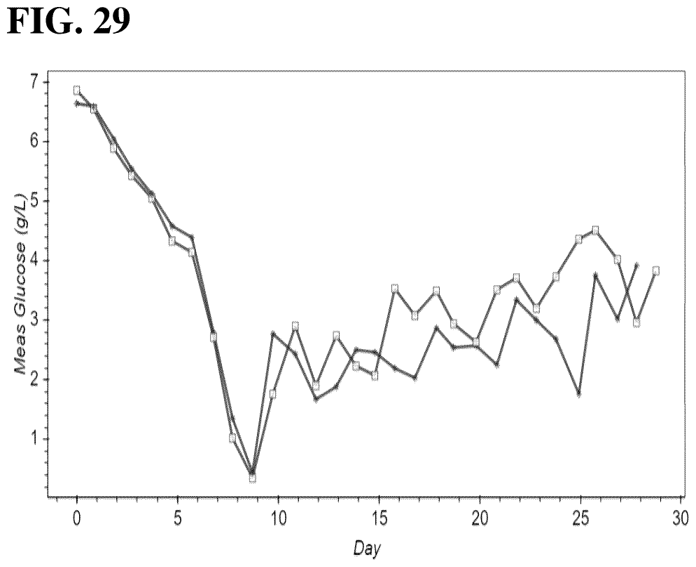

Glucose concentrations in the conditioned culture medium were low on day 9 as the cells reached or exceeded their target VCD (FIG. 29); however, glucose was never exhausted, and the supplemental drip kept the glucose concentration in a typical range for the rest of the run.

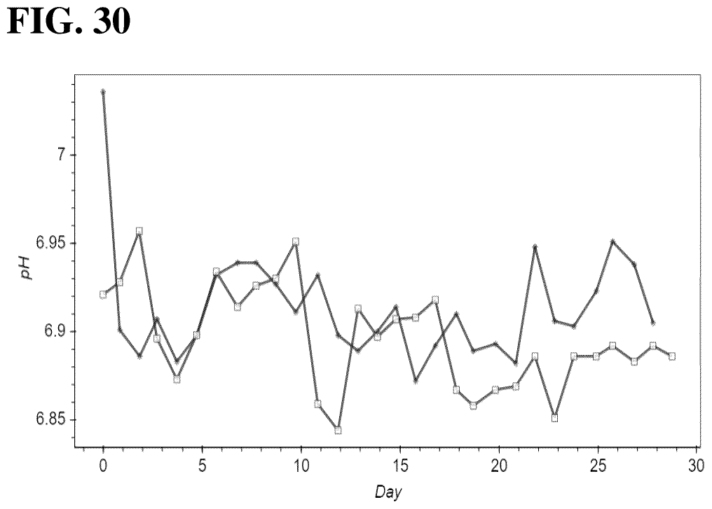

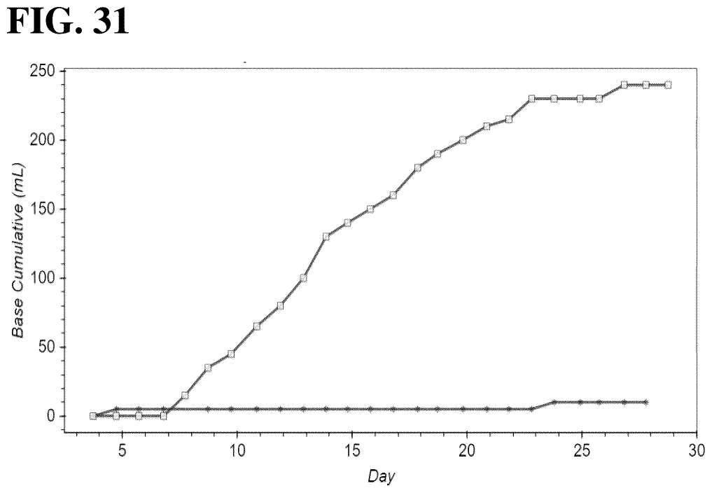

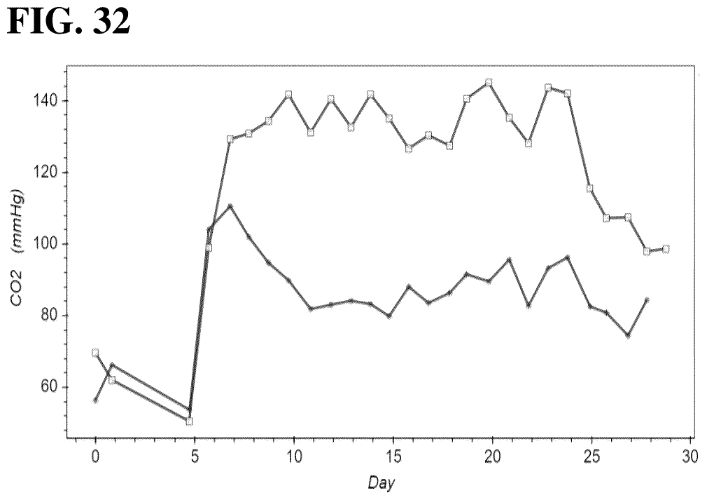

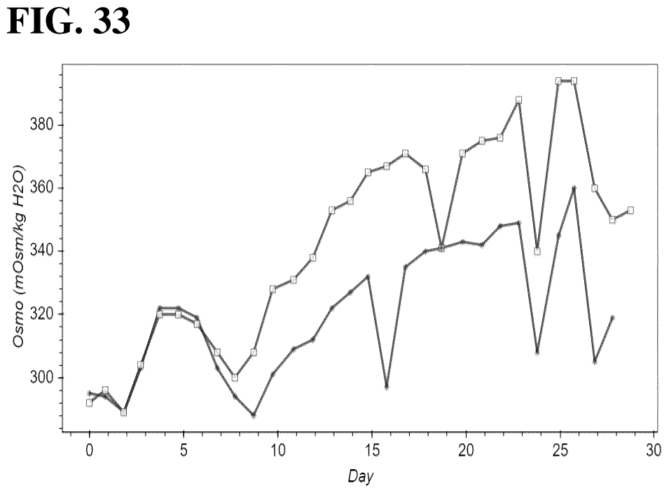

The pH of the culture medium remained within an acceptable range (FIG. 30). Base addition (FIG. 31) was highest for the unidirectional flow condition, which also exhibited higher CO₂ (FIG. 32) and sat consistently at the bottom of the pH deadband. It also had slightly higher osmolality (FIG. 33), corresponding to higher base addition. It is unclear why pH under the unidirectional flow condition differed from the bidirectional flow condition; however, both experimental conditions showed a typical range for CO₂ and base addition. Likewise, although osmolality climbed towards the end of the run, it remained within a typical range under both experimental conditions.

Comparing the unidirectional and bidirectional flow conditions of the TFF recirculation loop, the bidirectional flow condition's sieving and TMP were significantly better (about 20% higher sieving from about day 7 onward, and TMP did not increase until the last day of production), compared to the unidirectional flow condition. This showed that the inventive bidirectional TFF system benefited in superior filter performance across perfusion filter types that are commonly used for TFF perfusion in the biologics industry.

In addition, this experiment demonstrated that with the hollow fiber perfusion filter employed in this Example 2 (Spectrum® 0.2-μm pore size MiniKros® hollow-fiber filter with 0.047 m² surface area), TMP was also improved by the bidirectional flow condition, along with sieving. The bidirectional flow condition was also more productive than the unidirectional flow condition.

### Example 3. Testing Bidirectional TFF at Higher Cycle Times

An experiment was performed to test an embodiment of the centrifugal pump-based bidirectional TFF system described above. This embodiment used two Levitronix® TFF systems controlled remotely through Ignition (an integrated, flexible automation platform for supervisory control and data acquisition (SCADA); Inductive Automation) to periodically switch the direction of flow in the TFF recirculation loop on a predetermined cycle time. This system is schematically depicted in FIG. 1B to periodically switch direction of flow in the TFF recirculation loop on a predetermined cycle time and is described in Example 1 and Example 2, hereinabove. In this experiment, longer cycle times (6 hours and 12 hours) were used and compared to a 60-minute cycle time. The bidirectional conditions were compared with a unidirectional control.

Vial thaw, seed train expansion, and N−1 bioreactor operation were conducted as described in Example 1 and Example 2, hereinabove.

Production (N Stage) Bioreactor Operation. The production stage bioreactors were used to test the intensified protein production process with a high-performance cell line and an optimized intensified process. Productions were run in 1.6-L working volume glass and stainless-steel vessels using a typical perfusion filter and low-shear centrifugal TFF pump and controller. Operating parameters and setpoints are shown in Table 3.1, below.

TABLE 3.1
Production culture parameters and setpoints.

| Parameter | Value | |
|---|---|---|
| Cell Line/Molecule | Horizon CHO-K1 clone | |
| | producing an antibody | |
| Batch Medium | Ex-Cell Advanced CHO | |
| | Fed Batch Medium | |
| Bioreactor Type | Applikon glass w/stainless | |
| | parts (autoclavable) | |
| Digital Control Unit | Applikon ADI 1010 | |
| pH | 6.90 ± 0.02 | |
| Base type | 1M Sodium Carbonate | |
| Acid type | CO₂ gas | |
| Perfusion Media | Chemically-Defined Perfusion Media | |
| Perfusion Mode | Continuous Harvest | |
| Perfusion Recirculation Method | TFF | |
| Perfusion Filter | 0.09 m² Microza ® (Asahi Kasei) | |
| | with recycle | |
| Flux per day at 2VVD | 118.42 | L/m²/d |
| (Permeate + Recycle) | | |
| Recirculation flow sensor | Leviflow ® LFSC-D Series | |
| Pressure sensors | Pendotech PREPSN038 3/8″ | |
| | hose barb sensors | |
| Recirculation Flow Rate | 1.6 | L/min |
| (Crossflow) | | |
| Shear Inside Filter | 990 | s⁻¹ |
| Sintered Sparger Size | 15 | μm |
| Sparger Switch | When required - between | |
| Day | days 6-8 | |
| Temperature | 36.8° | C. |
| DO | 60% air saturation | |
| Agitation | 435 | RPM |
| Target Inoculation Cell Density | 6 × 10⁵ viable cells/mL | |
| Target VCD | 90e6 viable cells/mL | |
| Batch Working Volume | 1.25 L + 0.25 L to prime and | |
| | soak perfusion filter | |
| Nominal Working Volume | 1.6 | L |
| Air Overlay | 60 | sccm |
| Background Air Sparge | 60-175 | sccm |

| | |
|---|---|
| Background Air Sparge | Same as sparger switch |
| Start Day | day (see above) |

Bioreactor pH was controlled on one side of the deadband with sparged CO₂ through the same pipe as O₂ delivery, and on the other side of the deadband by addition of 1 M sodium carbonate. In-process 1-point calibration of pH was carried out if the offline pH measurement was more than 0.03 pH units from the probe value. Dissolved oxygen was maintained by pure O₂ delivered to the drilled hole pipe, then to a sintered sparger, when increased gas demand required it. A background air sparge delivered air through the drilled pipe throughout the run (starting on day 0) in order to strip CO₂. Starting mid-run, 50% glucose was added on an hourly timed drip using a peristaltic pump. The glucose feed was started on the day glucose dipped below 2.5 g/L when measured offline, and the pump rate was adjusted to maintain sufficient levels throughout the run. A continuous cell bleed was implemented mid-run to maintain cells at about 120×10⁶ cells/mL. Cell bleed was calculated independently of perfusion rate (such that a 10% bleed led to media addition equivalent to 2.1 vvd). Gamma-irradiated EX-CELL® Antifoam (SAFC) was added on an hourly drip using a timed pump. Antifoam addition started between days 2 and 5 starting at 0.2 mL/hour and was increased as needed to maintain a minimal head of foam on the culture.

Daily sampling was performed using a Nova BioProfile® FLEX2 for pH, CO₂, analytes such as lactate, glucose, and ammonium, and osmolality. Viable cell density (VCD), viability, and cell diameter snapshots were taken daily on a Vi-Cell XR. Packed cell volume (PCV) was measured regularly using specialized cuvettes. Finally, daily retains were saved from both the bioreactor and permeate; titer from these samples was used to calculate productivity (qP), and sieving throughout the run.

Perfusion operation was controlled as described in Example 1 hereinabove, except that the perfusion filter used for all conditions in this Example 3 was the Microza® 0.2-μm filter with 0.09 m² surface area (Asahi Kasei, Part No. UMP-1047R). For all conditions, a recycle pump was used to scale the total permeate flux through the filter to the equivalent flux of a 450-L working volume reactor with an appropriately-scaled filter, as described in Example 1, hereinabove. Table 2.2, below, shows the flux rates used to generate recycle rates for a 1.6-L working volume.

Perfusion was controlled for centrifugal TFF conditions (both bidirectional and unidirectional) using a Levitronix® pump console LCO-1100 and associated Levitronix® PuraLev®i100SU pump drive system(s).

For all conditions, a recycle pump was used to scale the total permeate flux through the filter to the equivalent flux of a 450 L working volume reactor with an appropriately-scaled filter. This was done by adding a two-pronged branching connector (i.e., a “Y”) to the permeate line; one arm of the “Y” was fed back into the pump at a continuous flow rate, while the other arm was fed into the permeate bag at the correct VVD. Table 3.2 (below) shows the flux rates used to generate recycle rates for a 1.6-L working volume.

TABLE 3.2
Perfusion ramp schedule.

| | Perfusion Rate, | Total Flux | Ideal | Ideal |
|---|---|---|---|---|
| | Vessel Volumes | per Day | Permeate Rate | Recycle Rate |
| Day | per Day (VVD) | (L/m2/d) | (mL/min) | (mL/min) |
| 0-2 | 0.0 | 0.00 | 0.00 | 0.00 |
| 2-3 | 0.5 | 29.61 | 0.56 | 0.41 |
| 3-4 | 1.0 | 59.21 | 1.12 | 0.82 |
| 4-5 | 1.5 | 88.82 | 1.68 | 1.23 |
| 5+ | 2.0 | 118.42 | 2.24 | 1.64 |

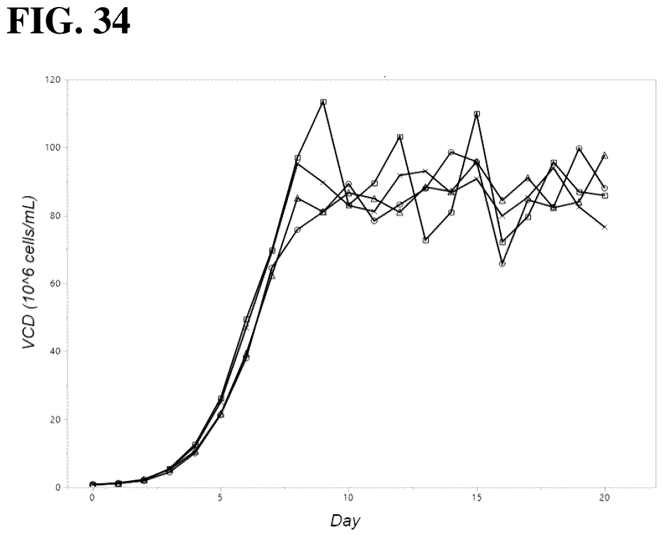

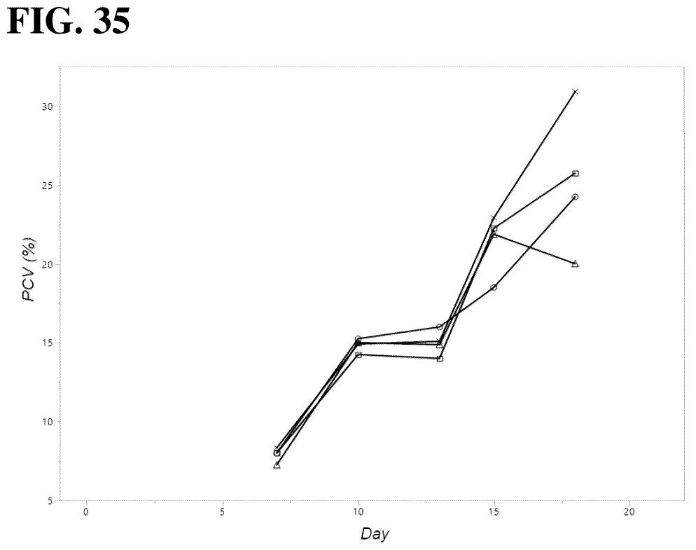

Results and Discussion. This experimental run was terminated on day 20 due to significant filter fouling that led to the reactors not being able to maintain the 2VVD perfusion rate setpoint. Overall, all conditions grew well, with VCDs reaching target density (90×10⁶ cells/mL) around day 8-10 and remaining there consistently with a small cell bleed until the end of the run (FIG. 34). Packed cell volume (PCV; see, FIG. 35) was also consistent across conditions, although the unidirectional condition had an ending PCV significantly higher than the other conditions.

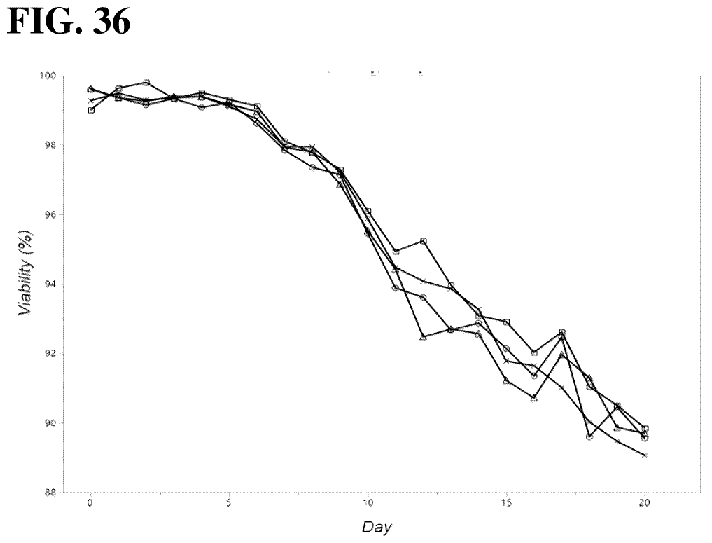

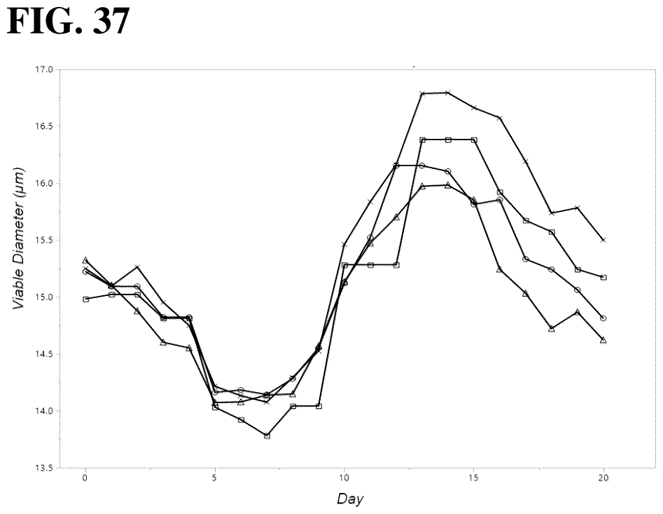

All conditions followed a similar viability trend (FIG. 36). Viability remained greater than 87% throughout the run for all conditions. Corresponding with the slightly higher PCV in the unidirectional condition, this condition also had a slightly higher viable cell diameter towards the end of the run (FIG. 37). Cell diameter followed a typical trend, similar to previous experiments (see, Example 1 and Example 2, above).

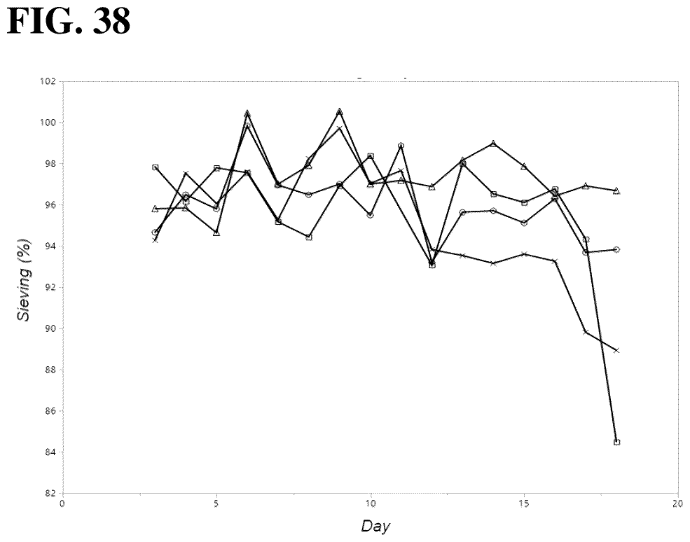

Sieving data were collected between days 3 and 18 (FIG. 38). Overall, sieving remained high for all conditions until about day 13, when some divergence occurred. Most bidirectional conditions remained consistent and greater than 93% until the end of the run, while the unidirectional condition dropped between days 13 and 18, ending at about 88%. Sieving for the 12-hour cycle time bidirectional condition was in line with the others until day 17, but sieving dropped to about 85% on the last day of culture.

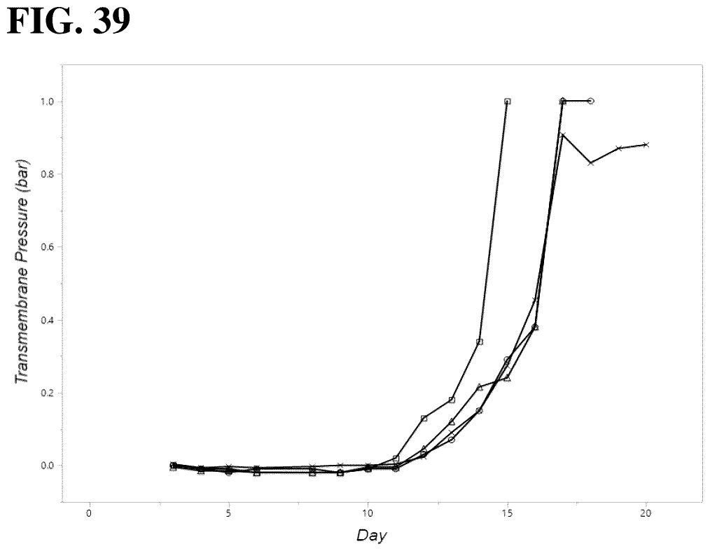

Transmembrane pressure (TMP) started increasing slightly sooner for the 12-hour bidirectional condition, compared to the other conditions; however, overall, TMP increased dramatically for all conditions around days 11-15 and remained higher for the rest of the run (FIG. 39).

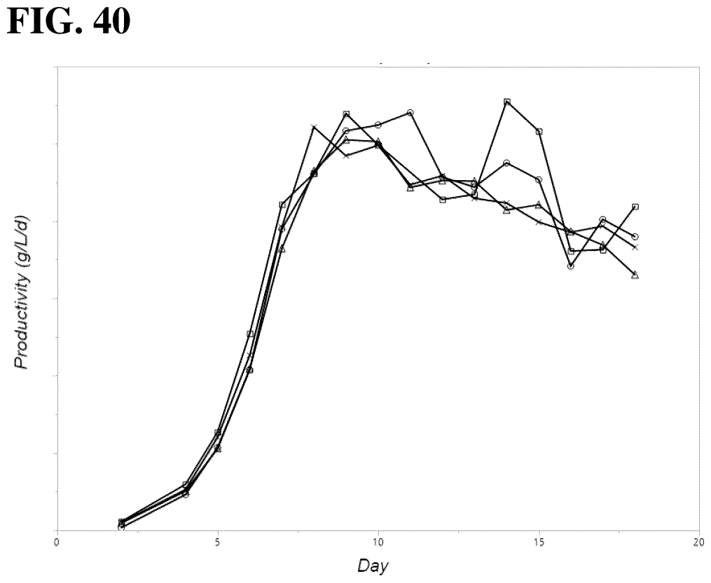

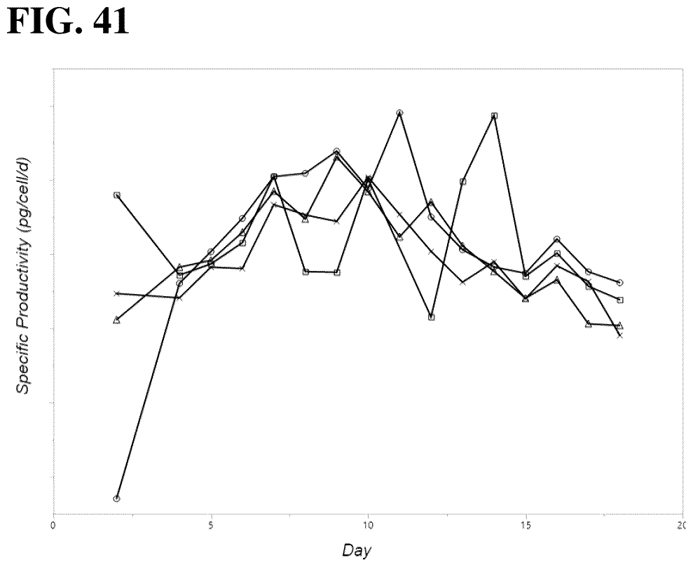

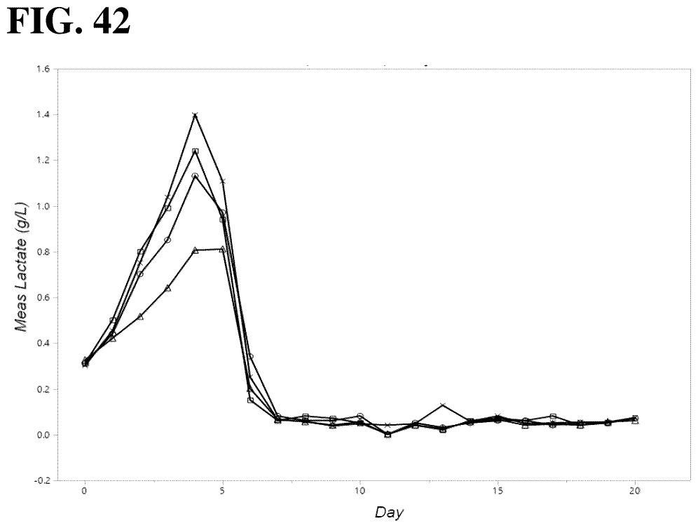

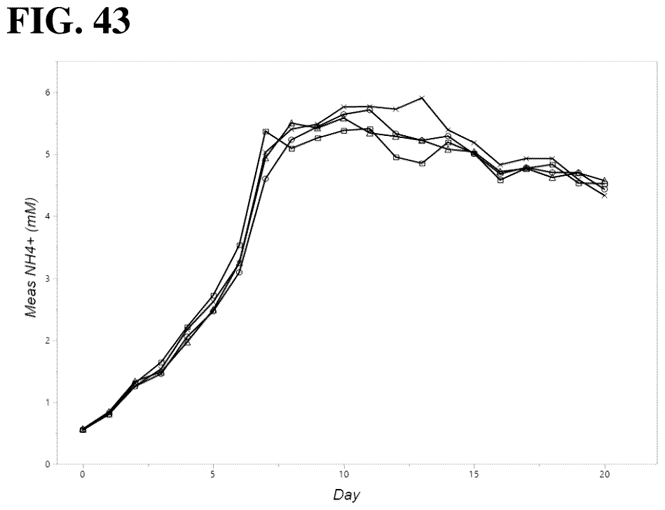

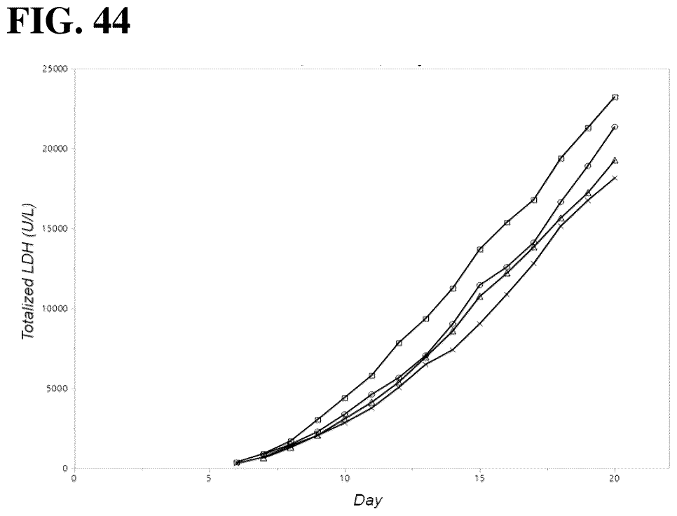

All conditions had similar antibody titer production (FIG. 40) and specific productivity (FIG. 41). Measured as in the previous examples, lactate concentration detected in the conditioned culture medium followed a typical profile, peaking towards the beginning of the run during the growth phase and then remaining very low for the rest of the run (FIG. 42). Ammonium concentration measured in the conditioned culture medium generally increased during the growth phase and then leveled off for the rest of the run in the typical range see in previous experiments with this process (FIG. 43). Totalized lactate dehydrogenase (LDH) enzyme measured in the conditioned culture medium (FIG. 44) was consistent throughout the run; LDH was slightly higher for the 12-hour bidirectional condition but similar for the other conditions.

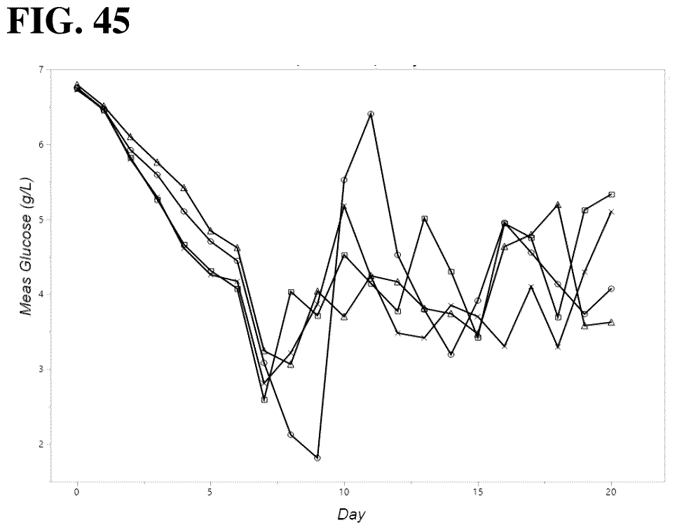

Glucose concentrations measured in the conditioned culture medium (FIG. 45) were kept within an acceptable range (mostly 2-7 g/L), decreasing as the cells reached their target density, but glucose concentrations were maintained with a supplemental glucose drip for the rest of the run.

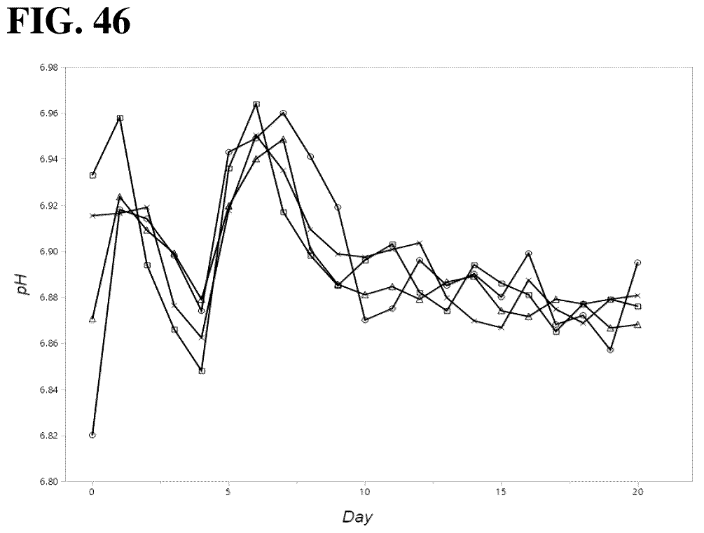

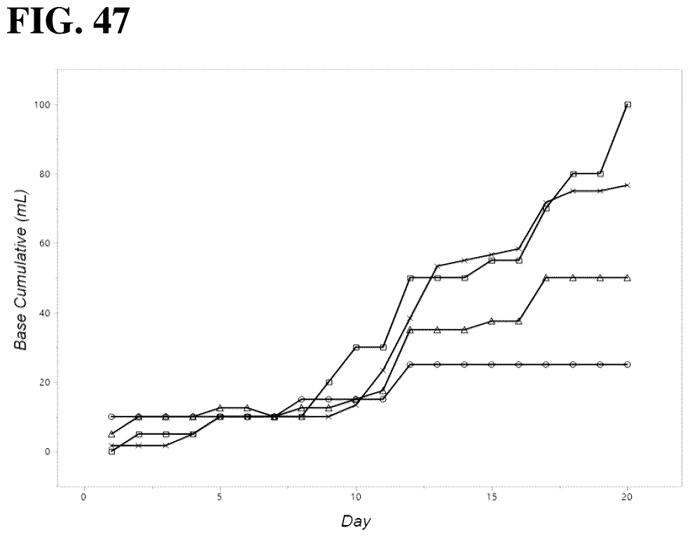

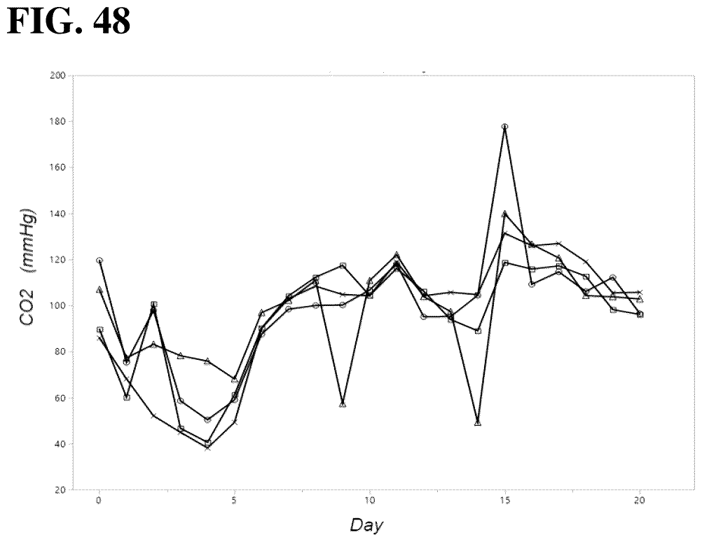

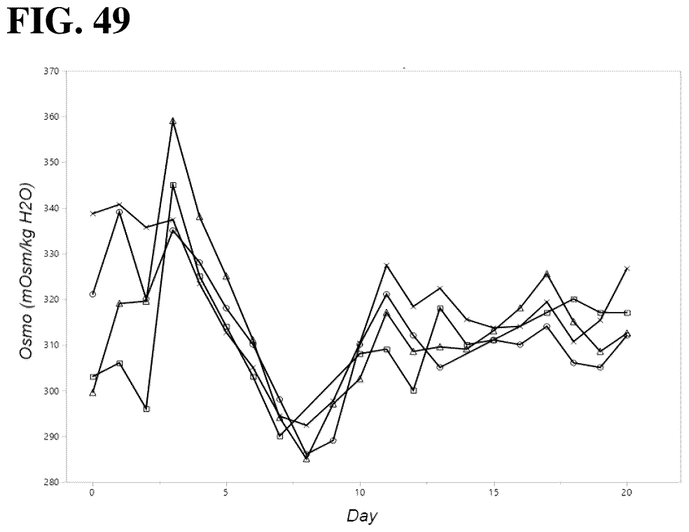

The pH measured in the conditioned culture medium remained within an acceptable range (FIG. 46). Cumulative base addition (FIG. 47), dissolved carbon dioxide (CO₂; FIG. 48), and osmolality measured in the conditioned culture medium (FIG. 49) also trended within typical profiles.

Although sieving differences were not as dramatic between the unidirectional and bidirectional conditions as demonstrated in Example 1-2 (above), the data from the experiment in this Example 3 demonstrate that the bidirectional automated tangential flow filtration (TFF) perfusion bioreactor of the invention provides improved sieving, compared to the unidirectional (standard) TFF conditions. The unidirectional condition trended lower between days 13 and 18 than the bidirectional flow conditions, excepting the 12-hour cycle time condition on its last day of culture. It is unclear what caused the drop in sieving or how much weight should be placed on this single point; even so, this bioreactor still had higher overall product recovery compared to the unidirectional condition.

These data imply that the benefits of the bidirectional TFF system may be more pronounced at shorter cycle times (e.g., 1-6 hours), and potentially at more extreme culture conditions (120×10⁶ cells/mL used in Examples 1 & 2, compared with the 90×10⁶ cells/mL used in Example 3) and longer culture durations. However, the smaller but still significant benefit seen here can translate to major gains in product recovery in large-scale biologics production processes.

The foregoing are merely exemplary, and the skilled practitioner of the present invention can easily vary the components and operating parameters as needed for a particular recombinant therapeutic protein drug substance of interest.

We claim:

1. An automated tangential flow filtration (TFF) perfusion bioreactor, comprising:
(a) a bioreactor configured for culturing eukaryotic cells, the bioreactor having an inlet for receiving volumes of fresh cell culture medium;
(b) a perfusion system comprising a recirculation loop fluidly linked with the bioreactor through a first port of the recirculation loop and a second port of the recirculation loop, the recirculation loop comprising, between the first port and the second port, a hollow fiber filter comprising a lumen having a porous surface capable of preventing the passage of whole cells but allowing volumes of conditioned cell culture medium to pass and to exit the perfusion system into a permeate receptacle, wherein volumes of conditioned medium containing cells from the bioreactor can enter through the first port into the recirculation loop, and flow tangentially in relation to the porous surface, and a retentate can flow out from the recirculation loop through the second port back into the bioreactor;
(c) a low-shear pump system comprising at least one pump head that directs the flow of the conditioned medium containing cells through the recirculation loop in a first direction across the porous surface of the hollow fiber filter and, interchangeably, in a second direction, opposite the first direction; and
(d) a controller capable of automatically switching the low-shear pump system to direct the flow of the conditioned medium containing cells through the recirculation loop in the first direction, and after a predetermined period, automatically switching the low-shear pump system to direct the flow of the conditioned medium containing cells to the second direction, and vice versa, wherein the first and the second directions are periodically exchanged and the conditioned medium containing cells synchronously enters the recirculation loop from the bioreactor through the second port instead of the first port and the retentate returns to the bioreactor through the first port instead of the second port, and vice versa;
wherein the TFF perfusion bioreactor is configured to operate over a production cultivation period of 13-90 days at a viable cell density of about 60-300×10⁶/mL, and wherein sieving is improved when compared to an equivalently-scaled non-switching system.

2. The automated tangential flow filtration perfusion bioreactor of claim 1, wherein the low shear pump system comprises one or more centrifugal pumps.

3. The automated tangential flow filtration perfusion bioreactor of claim 1, wherein the controller is configured to automatically switch the direction of flow through the low-shear pump system after the predetermined period of about 20 minutes to about 24 hours.

4. The automated tangential flow filtration perfusion bioreactor of claim 1, wherein the perfusion bioreactor is configured for culturing mammalian cells, insect cells, algal cells, or yeast cells.

5. The automated tangential flow filtration perfusion bioreactor of claim 1, wherein the perfusion bioreactor is configured for culturing mammalian cells.

6. The automated tangential flow filtration perfusion bioreactor of claim 1, wherein the TFF perfusion bioreactor is configured to operate over a production cultivation period of 20-60 days.

7. The automated tangential flow filtration perfusion bioreactor of claim 1, wherein the TFF perfusion bioreactor is configured to operate at a viable cell density of about 100-150×10⁶/mL.

8. The automated tangential flow filtration perfusion bioreactor of claim 1, wherein the bioreactor is configured for single-use.

9. The automated tangential flow filtration perfusion bioreactor of claim 1, wherein the recirculation loop is configured for single-use.
